SEQUENCE LISTING (120 sequences)

SEQ ID NO: 1 - DNA, 951 bp, Escherichia coli
  CDS (1)..(951)
atgtcaaagagaaaagttgctattatcggtagtggaaacatcggtactgatttgatgatcaagatccttagacatggacaacacttggaaatggctgttatggtcggtatcgatcctcagtcagacggacttgctagagccagaagaatgggtgttgctactacacatgaaggtgttattggattgatgaacatgccagagtttgcagatattgacatcgttttcgatgctacaagtgcaggtgctcacgttaagaatgacgctgccttgagagaagctaaacctgatattagattgatcgaccttaccccagcagctattggaccatactgtgttcctgttgtcaacttggaggccaatgttgatcaacttaacgttaatatggtcacatgcggtggacaggctaccattcctatggttgccgcagtctctagagttgctagagtccattatgccgaaattatcgcatccatcgcttcaaagagtgcaggtccaggaaccagagctaacattgacgaattcactgagaccacttctagagctatcgaagttgtcggtggagctgccaagggtaaagccattatcgttttgaatcctgcagagccacctcttatgatgagagatactgtttacgtcttgtctgacgaagcttcccaagatgacattgaggcctctatcaacgaaatggccgaggcagttcaggcttacgtcccaggttatagattgaagcaaagagttcagtttgaggtcattccacaagataaacctgttaatttgccaggtgtcggacagttctccggattgaaaacagctgtttggcttgaagtcgagggtgcagctcactacttgccagcttatgccggaaaccttgacattatgacttcttccgcattggctacagccgaaaagatggctcaatctcttgccagaaaagcaggagaggccgcataa

SEQ ID NO: 2 - protein, 316 aa, Escherichia coli
MSKRKVAIIGSGNIGTDLMIKILRHGQHLEMAVMVGIDPQSDGLARARRMGVATTHEGVIGLMNMPEFADIDIVFDATSAGAHVKNDAALREAKPDIRLIDLTPAAIGPYCVPVVNLEANVDQLNVNMVTCGGQATIPMVAAVSRVARVHYAEIIASIASKSAGPGTRANIDEFTETTSRAIEVVGGAAKGKAIIVLNPAEPPLMMRDTVYVLSDEASQDDIEASINEMAEAVQAYVPGYRLKQRVQFEVIPQDKPVNLPGVGQFSGLKTAVWLEVEGAAHYLPAYAGNLDIMTSSALATAEKMAQSLARKAGEAA

SEQ ID NO: 3 - DNA, 2676 bp, Escherichia coli
  CDS (1)..(2676)
atggcagttacgaacgttgcagagcttaatgcccttgtggagagggttaagaaagcacaaagggaatacgccagcttcacgcaagaacaagttgacaagattttcagggcagccgcgctagcggcagctgatgcgagaataccccttgctaagatggcagtcgcagaaagcggaatgggaattgtggaagataaggtcatcaagaatcatttcgcttctgagtatatctacaacgcgtataaggatgagaagacttgcggcgtcttatctgaggacgatacattcggcacaataactatcgctgaacctatagggatcatctgtggtatagttccaactacgaaccctacctcaactgctatattcaagagtcttatttctttgaaaacccgtaacgctatcatcttttctccacatccaagagccaaggacgccaccaacaaggcggcggacatcgtgctgcaggcggccatcgcggcgggcgcgcctaaggatctaattggctggatcgaccaacctagcgttgaactttccaatgctcttatgcaccatcctgatattaatttgatacttgcgactggaggccctgggatggtaaaggccgcttatagcagcggtaagcccgccatcggtgttggcgctggaaatacgccggtagtcattgatgagaccgccgacattaagagggcagtcgcgtcagttttgatgtctaagacttttgacaatggagtaatctgcgcgtcagagcagtccgttgtcgttgtggattcagtatacgacgcggtcagagaaaggttcgcgactcacggagggtacctacttcaagggaaggagttgaaagccgtccaggacgttatccttaagaatggtgcattgaacgccgccattgtaggccaaccagcgtataaaatagccgaacttgcgggctttagcgtaccagaaaacactaaaatacttattggtgaagtcaccgtggttgatgagtcagaacccttcgcccatgagaaactttccccaacactggcgatgtacagagcgaaggactttgaagatgcagtggaaaaagccgaaaagcttgttgcaatgggcggtatcggccacacaagctgcctttacacagatcaggataaccaaccagcaagagttagttatttcggccagaaaatgaagacggctaggatactaatcaacaccccggccagccaaggaggcattggtgacctgtataacttcaagttggctccttctcttacattgggctgtggttcctggggcggaaactctatctctgagaacgttggacctaagcatcttatcaataagaaaaccgtcgctaagcgtgctgagaacatgctttggcacaaactgccgaagtcaatctatttccgtaggggctccctgcctatcgcgttagatgaagtcatcaccgatgggcataagagagcactaatcgtcacagataggttcctattcaacaacggttacgcagaccaaatcacgtctgttctgaaagcggccggtgtcgaaacagaggtgttcttcgaagtggaagccgacccgacattaagtattgtcaggaaaggagctgagcttgcgaatagttttaagccggatgtcatcatcgctttgggaggaggatccccgatggatgctgctaagatcatgtgggtcatgtatgaacacccggagacacactttgaagagttggcgttgagattcatggatattaggaaaaggatctataaattccctaagatgggagtgaaagccaagatgatagcagtgacgaccaccagtggaaccgggagtgaagtgactccgtttgcggttgttactgacgacgctacgggccagaagtatcccctagccgactatgcattgacgccggatatggcgattgtggacgcgaacctggttatggatatgccaaagtcactttgcgcattcggtggacttgacgcagttacacatgcaatggaggcatatgtgtccgtgcttgcttcagagtttagcgatggacaggcacttcaagccttgaagttactaaaggaataccttcccgcatcttaccatgagggcagcaagaaccctgttgcccgtgagagagtacatagtgcggctaccattgccggtatcgcattcgctaatgctttcctgggtgtgtgtcattcaatggcccataagcttggatcccagtttcacatcccacacggattagccaacgctcttcttatatgtaacgtcatccgttataatgccaacgacaaccctacaaagcaaaccgcattctcacaatatgacaggccgcaggcgaggcgtcgttatgcagaaatagccgatcacctaggcttatccgcgccaggagatcgtacagctgctaagattgaaaagttgcttgcgtggttagaaacgcttaaggccgaactaggcattccaaagtctattagagaggcgggagttcaagaggcggatttccttgctaacgtggacaagcttagcgaagatgcattcgatgatcagtgcactggagccaacccgagatatcctctgatttcagaattgaagcaaattttacttgatacatactatgggagagactacgttgagggagagaccgcagcgaagaaggaggcggcgccggctaaggccgagaagaaagcaaagaaatcagcttaa

SEQ ID NO: 4 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 5 - DNA, 1404 bp, Escherichia coli
  CDS (1)..(1404)
atgaaccaacaagacatagaacaagtagtaaaggcagtattattaaagatgcaatcctctgacacaccaccagccgcagtacacgaaatgggtgtatttgcctctttggatgacgctgttgctgcagccaaaatagctcaacaaggtttgaagtcagttgcaatgagacaattagccatcgctgcaattagagaagctggtgaaaaacatgcaagagatttggccgaattagctgtctccgaaaccggtatgggtagagtagaagacaaattcgctaagaatgttgctcaagcaagaggtactccaggtgttgaatgtttgagtcctcaagtcttaactggtgacaacggtttgacattgatcgaaaacgcaccatggggtgttgtcgcctctgttactccatcaacaaatcctgccgctactgtcatcaataacgctatatctttgatcgcagccggtaactcagttatttttgcaccacatcctgctgctaagaaagtttcccaaagagctatcacattgttgaaccaagcaatcgttgccgctggtggtccagaaaatttgttagtcaccgtagccaaccctgatatagaaactgcacaaagattgttcaagttccctggtatcggtttgttagtagttacaggtggtgaagctgtcgtagaagcagccagaaaacacaccaataagagattgattgctgcaggtgctggtaacccacctgttgtcgtagatgaaactgcagacttagccagagccgctcaatccattgttaagggtgctagtttcgataacaacataatatgcgcagacgaaaaggtattgatagttgtcgattctgttgctgacgaattgatgagattaatggaaggtcaacatgcagttaaattgactgctgaacaagcacaacaattgcaaccagttttgttgaagaacatagatgaaagaggcaagggtacagtctcaagagattgggttggtagagacgctggcaagattgcagccgctataggtttaaaggtcccacaagaaactagattgttgttcgtagaaactacagccgaacatcctttcgctgtcacagaattgatgatgccagtattacctgtagttagagtagctaatgttgccgatgctatcgcattggccgttaaattagaaggtggttgtcatcacacagcagccatgcactccagaaacatcgaaaacatgaaccaaatggctaacgcaatcgacaccagtatttttgttaagaacggtccatgcatagctggtttgggtttaggtggtgaaggttggaccactatgacaatcacaacccctaccggtgaaggtgttacctctgctagaacttttgtcagattgagaagatgtgttttagtcgatgcattcagaattgtttag

SEQ ID NO: 6 - protein, 467 aa, Escherichia coli
MNQQDIEQVVKAVLLKMQSSDTPPAAVHEMGVFASLDDAVAAAKIAQQGLKSVAMRQLAIAAIREAGEKHARDLAELAVSETGMGRVEDKFAKNVAQARGTPGVECLSPQVLTGDNGLTLIENAPWGVVASVTPSTNPAATVINNAISLIAAGNSVIFAPHPAAKKVSQRAITLLNQAIVAAGGPENLLVTVANPDIETAQRLFKFPGIGLLVVTGGEAVVEAARKHTNKRLIAAGAGNPPVVVDETADLARAAQSIVKGASFDNNIICADEKVLIVVDSVADELMRLMEGQHAVKLTAEQAQQLQPVLLKNIDERGKGTVSRDWVGRDAGKIAAAIGLKVPQETRLLFVETTAEHPFAVTELMMPVLPVVRVANVADAIALAVKLEGGCHHTAAMHSRNIENMNQMANAIDTSIFVKNGPCIAGLGLGGEGWTTMTITTPTGEGVTSARTFVRLRRCVLVDAFRIV

SEQ ID NO: 7 - DNA, 1407 bp, Clostridium beijerinckii
  CDS (1)..(1407)
atgaataaggataccttgattccaactacaaaggatttgaaggttaagacaaacggtgaaaacatcaacttgaaaaattacaaggataactcttcatgtttcggtgttttcgaaaacgttgaaaacgctatttccagtgctgttcatgcacaaaagattttgtcattgcattacactaaagaacaaagagaaaagattatcacagaaatcagaaaggctgcattgcaaaataaggaagttttggcaaccatgatcttggaagaaactcatatgggtagatacgaagataagatcttgaagcatgaattagttgctaaatacacaccaggtactgaagatttgaccactacagcatggtccggtgacaatggtttaactgttgttgaaatgagtccttatggtgttattggtgctattactccatctacaaatcctaccgaaactgttatttgtaactcaatcggtatgattgctgctggtaatgctgttgtttttaacggtcatccatgtgctaagaaatgtgttgctttcgcagttgaaatgatcaataaggctatcatctcttgtggtggtccagaaaatttggttaccactattaagaaccctactatggaatctttggatgcaattattaagcatccatcaattaagttgttatgtggtacaggtggtcctggtatggttaagaccttgttgaactctggtaaaaaggctattggtgctggtgcaggtaacccacctgttattgttgatgatacagctgatatcgaaaaggcaggtagatctatcatcgaaggttgttcattcgataacaacttgccttgtatcgctgaaaaggaagttttcgttttcgaaaacgttgcagatgatttgatctccaacatgttaaagaataacgctgttattattaatgaagatcaagtttcaaagttaattgatttggttttacaaaagaataacgaaactcaagaatacttcatcaataagaaatgggttggtaaagatgctaagttgttcttagatgaaatcgatgttgaatctccatcaaacgttaagtgtatcatctgtgaagttaatgcaaaccatccttttgttatgacagaattgatgatgccaatcttgcctatcgttagagttaaggatattgatgaagctattaaatacgctaagatcgcagaacaaaacagaaagcattccgcatacatctatagtaagaacatcgataatttgaacagatttgaaagagaaatcgatacaaccatcttcgttaaaaatgctaagtctttcgcaggtgttggttacgaagctgaaggttttactacattcaccattgcaggttccactggtgaaggtattacaagtgctagaaacttcactagacaaagaagatgtgttttagcaggttaa

SEQ ID NO: 8 - protein, 468 aa, Clostridium beijerinckii
MNKDTLIPTTKDLKVKTNGENINLKNYKDNSSCFGVFENVENAISSAVHAQKILSLHYTKEQREKIITEIRKAALQNKEVLATMILEETHMGRYEDKILKHELVAKYTPGTEDLTTTAWSGDNGLTVVEMSPYGVIGAITPSTNPTETVICNSIGMIAAGNAVVFNGHPCAKKCVAFAVEMINKAIISCGGPENLVTTIKNPTMESLDAIIKHPSIKLLCGTGGPGMVKTLLNSGKKAIGAGAGNPPVIVDDTADIEKAGRSIIEGCSFDNNLPCIAEKEVFVFENVADDLISNMLKNNAVIINEDQVSKLIDLVLQKNNETQEYFINKKWVGKDAKLFLDEIDVESPSNVKCIICEVNANHPFVMTELMMPILPIVRVKDIDEAIKYAKIAEQNRKHSAYIYSKNIDNLNRFEREIDTTIFVKNAKSFAGVGYEAEGFTTFTIAGSTGEGITSARNFTRQRRCVLAG

SEQ ID NO: 9 - DNA, 2865 bp, Chlamydomonas reinhardtii
  CDS (1)..(2865)
atgatgagttcctctctggttagtggtaagagagttgccgtaccttctgccgcaaaaccgtgcgctgcagttcctctacctagggttgctggcagaagaacggctgcaagagttgtgtgcgaagcagcgccttcaggtgctgcccctgcctctccaaaagcagaggcagctgctcctgttgcagctgctcccgctacacctcatgcagaagttaagaaagaaagagcgccagcaactgacgaagcgttgacagagttaaaggctttgctaaagagagcacaaactgcacaagctcagtattctacatacactcaagaacaggtcgacgaaatctttcgtgcagccgctgaagcagctaacgcagccagaatacctttggccaaaatggcagtggaagaaactcgtatgggagtggctgaagataaagttgttaagaatcactttgcgtcagaattcatttataataagtataaacacacgaaaacttgtggtgtgatcgagcatgacccagcaggagggattcagaaagtggctgagcctgtaggtgtaatagctggtatcgttccaactactaatcccacttctacggcaatatttaagtccctactaagcttaaagaccagaaacgccttggttttgtgtccccaccctagggcagctaaaagtacaatcgcagctgccagaatagtgagagatgcggctgtcgcagctggagctcctcccaatataatttcttgggtggaaacaccgtcgttgccagtaagtcaagccttgatgcaagctactgaaattaacttgattttagctactggtggtcccgccatggtaagagctgcatacagttcagggaatccttctttgggagttggcgctgggaatacaccggctttgatagatgaaaccgcagatgtagccatggcagtctcttccattctactgagcaaaaccttcgacaatggtgtgatttgtgcttcagaacaaagcgttgtggtcgttgccaaggcttatgacgctgtcaggacagaattcgttaggagaggtgcctactttctaacggaagatgataaggtaaaggttagagcgggagtcgtagtagatggcaaattgaatccaaatatagttggccagtcaattccaaaacttgccgccctatttggcataaaggttcctcaaggcacaaaagttttgattggtgaggtagagaaaatcggtccagaagaagctttatctcaagagaaactttgccccatactggcaatgtatagagctccagattacgaccatggtgtcaaaatggcttgtgagttaataatgtacggtggtgccggacatacgagcgttttgtacaccaatccccttaataatgcgcacattcagcagtatcaatcagctgtcaaaacagtgagaatcttgattaacactccagctagtcaaggtgcaatcggtgatctttacaattttcatcttgatccctctcttacattgggctgtggtacatggggctcaacttctgtatcaacgaatgttggtccccaacatcttttgaacatcaaaaccgtaacagccaggagggaaaacatgttatggtttcgtgttccacctaaaatctactttaaaggtggatgtttagaagttgcgttaaccgacctgagagggaaaagcagagctttcattgtaactgataagccattatttgacatgggttatgctgataaagtcacgcacattttagactccattaacgtgcatcatcaagttttctaccacgttacaccggaccctacattggcttgcattgaagcggggttaaaagagatcttggaatttaaaccagacgttataatcgctttaggcggagggagtccaatggatgccgctaagatcatgtggctaatgtacgaatgcccagacaccagatttgatggactagccatgaggttcatggatattagaaagagagtttatgaggttccggaattgggcaagaaagcaaccatggtgtgcattcctacaacctctggtacaggttccgaagtcactccattttctgtagttacagatgaacgtttgggcgcaaaatatccactggcagattatgcattgactccgtcgatggccattgtcgatccacaattggtgttaaacatgcctaagaaattaaccgcctggggtggtattgacgcattgactcatgcgcttgagtcgtatgtctccatttgtgcgactgattatacgaagggattatcaagagaagccatatcactactgttcaagtacttgcccagggcatatgcaaacgggtcgaatgattacttagctagagaaaaggttcactatgccgcgactattgctggtatggcctttgcaaacgcattcttaggaatatgtcactccatggcgcataaattaggcgctgcttatcatgtgcctcatggtctagctaatgcagccttaatttcgcatgtcatacgttataacgctaccgatatgccagcgaaacaagccgctttcccacaatacgagtatcctactgcaaagcaagactatgctgatttggccaacatgctgggtcttggaggtaataccgtagatgagaaggtcatcaagttgattgaagcagtagaagaactgaaagctaaagttgacataccaccgactattaaggaaatctttaacgatccaaaggtggatgcggattttctggctaatgtcgacgctttggccgaggatgcatttgatgatcaatgtacaggagcaaatccaagatacccattgatggctgatttaaaacagttataccttgatgcccatgctgccccaattttaccagtcaaaacccttgaattcttctccaaaatcaactaa

SEQ ID NO: 10 - protein, 954 aa, Chlamydomonas reinhardtii
MMSSSLVSGKRVAVPSAAKPCAAVPLPRVAGRRTAARVVCEAAPSGAAPASPKAEAAAPVAAAPATPHAEVKKERAPATDEALTELKALLKRAQTAQAQYSTYTQEQVDEIFRAAAEAANAARIPLAKMAVEETRMGVAEDKVVKNHFASEFIYNKYKHTKTCGVIEHDPAGGIQKVAEPVGVIAGIVPTTNPTSTAIFKSLLSLKTRNALVLCPHPRAAKSTIAAARIVRDAAVAAGAPPNIISWVETPSLPVSQALMQATEINLILATGGPAMVRAAYSSGNPSLGVGAGNTPALIDETADVAMAVSSILLSKTFDNGVICASEQSVVVVAKAYDAVRTEFVRRGAYFLTEDDKVKVRAGVVVDGKLNPNIVGQSIPKLAALFGIKVPQGTKVLIGEVEKIGPEEALSQEKLCPILAMYRAPDYDHGVKMACELIMYGGAGHTSVLYTNPLNNAHIQQYQSAVKTVRILINTPASQGAIGDLYNFHLDPSLTLGCGTWGSTSVSTNVGPQHLLNIKTVTARRENMLWFRVPPKIYFKGGCLEVALTDLRGKSRAFIVTDKPLFDMGYADKVTHILDSINVHHQVFYHVTPDPTLACIEAGLKEILEFKPDVIIALGGGSPMDAAKIMWLMYECPDTRFDGLAMRFMDIRKRVYEVPELGKKATMVCIPTTSGTGSEVTPFSVVTDERLGAKYPLADYALTPSMAIVDPQLVLNMPKKLTAWGGIDALTHALESYVSICATDYTKGLSREAISLLFKYLPRAYANGSNDYLAREKVHYAATIAGMAFANAFLGICHSMAHKLGAAYHVPHGLANAALISHVIRYNATDMPAKQAAFPQYEYPTAKQDYADLANMLGLGGNTVDEKVIKLIEAVEELKAKVDIPPTIKEIFNDPKVDADFLANVDALAEDAFDDQCTGANPRYPLMADLKQLYLDAHAAPILPVKTLEFFSKIN

SEQ ID NO: 11 - DNA, 1320 bp, Unknown
  Obtained from Intestinal Protist of      Reticulitermes speratus
  CDS (1)..(1320)
atgtctcaaatttttaaggatatcccagttattaaatatgaaggtccagcttccaagaatcctttgagtttcaaatactacgatgcaaacaaggttattgatggtaaaccaatgaaggaacatttgagatacgcaatggcttggtggcataatttgtgtgctaccggtcaagatatgtttggtcctggtactgcagataaatccttcggtagtaagacagttggtaccatggaacatgcacatgctaaagttgatgctggttttgaattcatgtccaagttgggtgttgaatacttctgtttccatgatgctgatttggttccagaagcagatactttgagtgaaacaaacaaaagattggatgaaatcgctgaacatatcgttgctaagcaaaaggcaactggtattaaatgtttgtggggtacagcaaatttgttttctaaccctagattcttaaatggttctggttcttcaaactcagctgatgtttatgcatacgctgcagctcaaattaaaaaggctttggatttgactgttaaatttggtggtgttggttatgttttctggggtggtagagaaggttacgaaaccttgttgaacactgatgttaagttcgaacaagaaaacatcgctaacttgatgcatttggcagttacttacggtagatcaatcggttttaaaggtgacttctacattgaaccaaaacctaaggaaccaacaaagcatcaatatgattttgatgcagctactacaattggtttcattagacaatacggtttggaaaaggatttcaagttgaacatcgaagcaaaccatgctacattagcaggtcataccttccaacatgatttgagaatctctgctattaatggcatgttaggttcagttgatgcaaacacaggtgacccattgttaggttgggataccgatgaatttccttattccgtttacgataccactttggctatgtacgaaattattaaggcaggtggtttgaccggtggtttgaattttgattccaaggttagaagaccaagttacacacatgaagatttgttttacggtttcattttgggtatggattctttcgctttgggtttgattaaagcaaaggctttgattgcagatggtagattggattcattcgttaaggatagatacgcttcttacggttcaggtattggtgctaagattagagatcattctgcaactttggaagaattagcagcttatgcattagctaaagatacagttgctttgcctggttccggtagacaagaatacttagaaagtattattaaccaaattttgtttcaataa

SEQ ID NO: 12 - protein, 439 aa, Unknown
  Synthetic Construct
MSQIFKDIPVIKYEGPASKNPLSFKYYDANKVIDGKPMKEHLRYAMAWWHNLCATGQDMFGPGTADKSFGSKTVGTMEHAHAKVDAGFEFMSKLGVEYFCFHDADLVPEADTLSETNKRLDEIAEHIVAKQKATGIKCLWGTANLFSNPRFLNGSGSSNSADVYAYAAAQIKKALDLTVKFGGVGYVFWGGREGYETLLNTDVKFEQENIANLMHLAVTYGRSIGFKGDFYIEPKPKEPTKHQYDFDAATTIGFIRQYGLEKDFKLNIEANHATLAGHTFQHDLRISAINGMLGSVDANTGDPLLGWDTDEFPYSVYDTTLAMYEIIKAGGLTGGLNFDSKVRRPSYTHEDLFYGFILGMDSFALGLIKAKALIADGRLDSFVKDRYASYGSGIGAKIRDHSATLEELAAYALAKDTVALPGSGRQEYLESIINQILFQ

SEQ ID NO: 13 - DNA, 1047 bp, Saccharomyces cerevisiae
  CDS (1)..(1047)
atgtctatcccagaaactcaaaaaggtgttatcttctacgaatcccacggtaagttggaatacaaagatattccagttccaaagccaaaggccaacgaattgttgatcaacgttaaatactctggtgtctgtcacactgacttgcacgcttggcacggtgactggccattgccagttaagctaccattagtcggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaacaatacgctaccgctgacgctgttcaagccgctcacattcctcaaggtaccgacttggcccaagtcgcccccatcttgtgtgctggtatcaccgtctacaaggctttgaagtctgctaacttgatggccggtcactgggttgctatctccggtgctgctggtggtctaggttctttggctgttcaatacgccaaggctatgggttacagagtcttgggtattgacggtggtgaaggtaaggaagaattattcagatccatcggtggtgaagtcttcattgacttcactaaggaaaaggacattgtcggtgctgttctaaaggccactgacggtggtgctcacggtgtcatcaacgtttccgtttccgaagccgctattgaagcttctaccagatacgttagagctaacggtaccaccgttttggtcggtatgccagctggtgccaagtgttgttctgatgtcttcaaccaagtcgtcaagtccatctctattgttggttcttacgtcggtaacagagctgacaccagagaagctttggacttcttcgccagaggtttggtcaagtctccaatcaaggttgtcggcttgtctaccttgccagaaatttacgaaaagatggaaaagggtcaaatcgttggtagatacgttgttgacacttctaaataa

SEQ ID NO: 14 - protein, 348 aa, Saccharomyces cerevisiae
MSIPETQKGVIFYESHGKLEYKDIPVPKPKANELLINVKYSGVCHTDLHAWHGDWPLPVKLPLVGGHEGAGVVVGMGENVKGWKIGDYAGIKWLNGSCMACEYCELGNESNCPHADLSGYTHDGSFQQYATADAVQAAHIPQGTDLAQVAPILCAGITVYKALKSANLMAGHWVAISGAAGGLGSLAVQYAKAMGYRVLGIDGGEGKEELFRSIGGEVFIDFTKEKDIVGAVLKATDGGAHGVINVSVSEAAIEASTRYVRANGTTVLVGMPAGAKCCSDVFNQVVKSISIVGSYVGNRADTREALDFFARGLVKSPIKVVGLSTLPEIYEKMEKGQIVGRYVVDTSK

SEQ ID NO: 15 - DNA, 1047 bp, Saccharomyces cerevisiae
  CDS (1)..(1047)
atgtctattccagaaactcaaaaagccattatcttctacgaatccaacggcaagttggagcataaggatatcccagttccaaagccaaagcccaacgaattgttaatcaacgtcaagtactctggtgtctgccacaccgatttgcacgcttggcatggtgactggccattgccaactaagttaccattagttggtggtcacgaaggtgccggtgtcgttgtcggcatgggtgaaaacgttaagggctggaagatcggtgactacgccggtatcaaatggttgaacggttcttgtatggcctgtgaatactgtgaattgggtaacgaatccaactgtcctcacgctgacttgtctggttacacccacgacggttctttccaagaatacgctaccgctgacgctgttcaagccgctcacattcctcaaggtactgacttggctgaagtcgcgccaatcttgtgtgctggtatcaccgtatacaaggctttgaagtctgccaacttgagagcaggccactgggcggccatttctggtgctgctggtggtctaggttctttggctgttcaatatgctaaggcgatgggttacagagtcttaggtattgatggtggtccaggaaaggaagaattgtttacctcgctcggtggtgaagtattcatcgacttcaccaaagagaaggacattgttagcgcagtcgttaaggctaccaacggcggtgcccacggtatcatcaatgtttccgtttccgaagccgctatcgaagcttctaccagatactgtagggcgaacggtactgttgtcttggttggtttgccagccggtgcaaagtgctcctctgatgtcttcaaccacgttgtcaagtctatctccattgtcggctcttacgtggggaacagagctgataccagagaagccttagatttctttgccagaggtctagtcaagtctccaataaaggtagttggcttatccagtttaccagaaatttacgaaaagatggagaagggccaaattgctggtagatacgttgttgacacttctaaataa

SEQ ID NO: 16 - protein, 348 aa, Saccharomyces cerevisiae
MSIPETQKAIIFYESNGKLEHKDIPVPKPKPNELLINVKYSGVCHTDLHAWHGDWPLPTKLPLVGGHEGAGVVVGMGENVKGWKIGDYAGIKWLNGSCMACEYCELGNESNCPHADLSGYTHDGSFQEYATADAVQAAHIPQGTDLAEVAPILCAGITVYKALKSANLRAGHWAAISGAAGGLGSLAVQYAKAMGYRVLGIDGGPGKEELFTSLGGEVFIDFTKEKDIVSAVVKATNGGAHGIINVSVSEAAIEASTRYCRANGTVVLVGLPAGAKCSSDVFNHVVKSISIVGSYVGNRADTREALDFFARGLVKSPIKVVGLSSLPEIYEKMEKGQIAGRYVVDTSK

SEQ ID NO: 17 - DNA, 1803 bp, Saccharomyces cerevisiae
  CDS (1)..(1803)
atgttgtgttcagtaattcagagacagacaagagaggtttccaacacaatgtctttagactcatactatcttgggtttgatctttcgacccaacaactgaaatgtctcgccattaaccaggacctaaaaattgtccattcagaaacagtggaatttgaaaaggatcttccgcattatcacacaaagaagggtgtctatatacacggcgacactatcgaatgtcccgtagccatgtggttagaggctctagatctggttctctcgaaatatcgcgaggctaaatttccattgaacaaagttatggccgtctcagggtcctgccagcagcacgggtctgtctactggtcctcccaagccgaatctctgttagagcaattgaataagaaaccggaaaaagatttattgcactacgtgagctctgtagcatttgcaaggcaaaccgcccccaattggcaagaccacagtactgcaaagcaatgtcaagagtttgaagagtgcataggtgggcctgaaaaaatggctcaattaacagggtccagagcccattttagatttactggtcctcaaattctgaaaattgcacaattagaaccagaagcttacgaaaaaacaaagaccatttctttagtgtctaattttttgacttctatcttagtgggccatcttgttgaattagaggaggcagatgcctgtggtatgaacctttatgatatacgtgaaagaaaattcagtgatgagctactacatctaattgatagttcttctaaggataaaactatcagacaaaaattaatgagagcacccatgaaaaatttgatagcgggtaccatctgtaaatattttattgagaagtacggtttcaatacaaactgcaaggtctctcccatgactggggataatttagccactatatgttctttacccctgcggaagaatgacgttctcgtttccctaggaacaagtactacagttcttctggtcaccgataagtatcacccctctccgaactatcatcttttcattcatccaactctgccaaaccattatatgggtatgatttgttattgtaatggttctttggcaagggagaggataagagacgagttaaacaaagaacgggaaaataattatgagaagactaacgattggactctttttaatcaagctgtgctagatgactcagaaagtagtgaaaatgaattaggtgtatattttcctctgggggagatcgttcctagcgtaaaagccataaacaaaagggttatcttcaatccaaaaacgggtatgattgaaagagaggtggccaagttcaaagacaagaggcacgatgccaaaaatattgtagaatcacaggctttaagttgcagggtaagaatatctcccctgctttcggattcaaacgcaagctcacaacagagactgaacgaagatacaatcgtgaagtttgattacgatgaatctccgctgcgggactacctaaataaaaggccagaaaggactttttttgtaggtggggcttctaaaaacgatgctattgtgaagaagtttgctcaagtcattggtgctacaaagggtaattttaggctagaaacaccaaactcatgtgcccttggtggttgttataaggccatgtggtcattgttatatgactctaataaaattgcagttccttttgataaatttctgaatgacaattttccatggcatgtaatggaaagcatatccgatgtggataatgaaaattgggatcgctataattccaagattgtccccttaagcgaactggaaaagactctcatctaa

SEQ ID NO: 18 - protein, 600 aa, Saccharomyces cerevisiae
MLCSVIQRQTREVSNTMSLDSYYLGFDLSTQQLKCLAINQDLKIVHSETVEFEKDLPHYHTKKGVYIHGDTIECPVAMWLEALDLVLSKYREAKFPLNKVMAVSGSCQQHGSVYWSSQAESLLEQLNKKPEKDLLHYVSSVAFARQTAPNWQDHSTAKQCQEFEECIGGPEKMAQLTGSRAHFRFTGPQILKIAQLEPEAYEKTKTISLVSNFLTSILVGHLVELEEADACGMNLYDIRERKFSDELLHLIDSSSKDKTIRQKLMRAPMKNLIAGTICKYFIEKYGFNTNCKVSPMTGDNLATICSLPLRKNDVLVSLGTSTTVLLVTDKYHPSPNYHLFIHPTLPNHYMGMICYCNGSLARERIRDELNKERENNYEKTNDWTLFNQAVLDDSESSENELGVYFPLGEIVPSVKAINKRVIFNPKTGMIEREVAKFKDKRHDAKNIVESQALSCRVRISPLLSDSNASSQQRLNEDTIVKFDYDESPLRDYLNKRPERTFFVGGASKNDAIVKKFAQVIGATKGNFRLETPNSCALGGCYKAMWSLLYDSNKIAVPFDKFLNDNFPWHVMESISDVDNENWDRYNSKIVPLSELEKTLI

SEQ ID NO: 19 - DNA, 2043 bp, Saccharomyces cerevisiae
  CDS (1)..(2043)
atgactcaattcactgacattgataagctagccgtctccaccataagaattttggctgtggacaccgtatccaaggccaactcaggtcacccaggtgctccattgggtatggcaccagctgcacacgttctatggagtcaaatgcgcatgaacccaaccaacccagactggatcaacagagatagatttgtcttgtctaacggtcacgcggtcgctttgttgtattctatgctacatttgactggttacgatctgtctattgaagacttgaaacagttcagacagttgggttccagaacaccaggtcatcctgaatttgagttgccaggtgttgaagttactaccggtccattaggtcaaggtatctccaacgctgttggtatggccatggctcaagctaacctggctgccacttacaacaagccgggctttaccttgtctgacaactacacctatgttttcttgggtgacggttgtttgcaagaaggtatttcttcagaagcttcctccttggctggtcatttgaaattgggtaacttgattgccatctacgatgacaacaagatcactatcgatggtgctaccagtatctcattcgatgaagatgttgctaagagatacgaagcctacggttgggaagttttgtacgtagaaaatggtaacgaagatctagccggtattgccaaggctattgctcaagctaagttatccaaggacaaaccaactttgatcaaaatgaccacaaccattggttacggttccttgcatgccggctctcactctgtgcacggtgccccattgaaagcagatgatgttaaacaactaaagagcaaattcggtttcaacccagacaagtcctttgttgttccacaagaagtttacgaccactaccaaaagacaattttaaagccaggtgtcgaagccaacaacaagtggaacaagttgttcagcgaataccaaaagaaattcccagaattaggtgctgaattggctagaagattgagcggccaactacccgcaaattgggaatctaagttgccaacttacaccgccaaggactctgccgtggccactagaaaattatcagaaactgttcttgaggatgtttacaatcaattgccagagttgattggtggttctgccgatttaacaccttctaacttgaccagatggaaggaagcccttgacttccaacctccttcttccggttcaggtaactactctggtagatacattaggtacggtattagagaacacgctatgggtgccataatgaacggtatttcagctttcggtgccaactacaaaccatacggtggtactttcttgaacttcgtttcttatgctgctggtgccgttagattgtccgctttgtctggccacccagttatttgggttgctacacatgactctatcggtgtcggtgaagatggtccaacacatcaacctattgaaactttagcacacttcagatccctaccaaacattcaagtttggagaccagctgatggtaacgaagtttctgccgcctacaagaactctttagaatccaagcatactccaagtatcattgctttgtccagacaaaacttgccacaattggaaggtagctctattgaaagcgcttctaagggtggttacgtactacaagatgttgctaacccagatattattttagtggctactggttccgaagtgtctttgagtgttgaagctgctaagactttggccgcaaagaacatcaaggctcgtgttgtttctctaccagatttcttcacttttgacaaacaacccctagaatacagactatcagtcttaccagacaacgttccaatcatgtctgttgaagttttggctaccacatgttggggcaaatacgctcatcaatccttcggtattgacagatttggtgcctccggtaaggcaccagaagtcttcaagttcttcggtttcaccccagaaggtgttgctgaaagagctcaaaagaccattgcattctataagggtgacaagctaatttctcctttgaaaaaagctttctaa

SEQ ID NO: 20 - protein, 680 aa, Saccharomyces cerevisiae
MTQFTDIDKLAVSTIRILAVDTVSKANSGHPGAPLGMAPAAHVLWSQMRMNPTNPDWINRDRFVLSNGHAVALLYSMLHLTGYDLSIEDLKQFRQLGSRTPGHPEFELPGVEVTTGPLGQGISNAVGMAMAQANLAATYNKPGFTLSDNYTYVFLGDGCLQEGISSEASSLAGHLKLGNLIAIYDDNKITIDGATSISFDEDVAKRYEAYGWEVLYVENGNEDLAGIAKAIAQAKLSKDKPTLIKMTTTIGYGSLHAGSHSVHGAPLKADDVKQLKSKFGFNPDKSFVVPQEVYDHYQKTILKPGVEANNKWNKLFSEYQKKFPELGAELARRLSGQLPANWESKLPTYTAKDSAVATRKLSETVLEDVYNQLPELIGGSADLTPSNLTRWKEALDFQPPSSGSGNYSGRYIRYGIREHAMGAIMNGISAFGANYKPYGGTFLNFVSYAAGAVRLSALSGHPVIWVATHDSIGVGEDGPTHQPIETLAHFRSLPNIQVWRPADGNEVSAAYKNSLESKHTPSIIALSRQNLPQLEGSSIESASKGGYVLQDVANPDIILVATGSEVSLSVEAAKTLAAKNIKARVVSLPDFFTFDKQPLEYRLSVLPDNVPIMSVEVLATTCWGKYAHQSFGIDRFGASGKAPEVFKFFGFTPEGVAERAQKTIAFYKGDKLISPLKKAF

SEQ ID NO: 21 - DNA, 1008 bp, Saccharomyces cerevisiae
  CDS (1)..(1008)
atgtctgaaccagctcaaaagaaacaaaaggttgctaacaactctctagaacaattgaaagcctccggcactgtcgttgttgccgacactggtgatttcggctctattgccaagtttcaacctcaagactccacaactaacccatcattgatcttggctgctgccaagcaaccaacttacgccaagttgatcgatgttgccgtggaatacggtaagaagcatggtaagaccaccgaagaacaagtcgaaaatgctgtggacagattgttagtcgaattcggtaaggagatcttaaagattgttccaggcagagtctccaccgaagttgatgctagattgtcttttgacactcaagctaccattgaaaaggctagacatatcattaaattgtttgaacaagaaggtgtctccaaggaaagagtccttattaaaattgcttccacttgggaaggtattcaagctgccaaagaattggaagaaaaggacggtatccactgtaatttgactctattattctccttcgttcaagcagttgcctgtgccgaggcccaagttactttgatttccccatttgttggtagaattctagactggtacaaatccagcactggtaaagattacaagggtgaagccgacccaggtgttatttccgtcaagaaaatctacaactactacaagaagtacggttacaagactattgttatgggtgcttctttcagaagcactgacgaaatcaaaaacttggctggtgttgactatctaacaatttctccagctttattggacaagttgatgaacagtactgaacctttcccaagagttttggaccctgtctccgctaagaaggaagccggcgacaagatttcttacatcagcgacgaatctaaattcagattcgacttgaatgaagacgctatggccactgaaaaattgtccgaaggtatcagaaaattctctgccgatattgttactctattcgacttgattgaaaagaaagttaccgcttaa

SEQ ID NO: 22 - protein, 335 aa, Saccharomyces cerevisiae
MSEPAQKKQKVANNSLEQLKASGTVVVADTGDFGSIAKFQPQDSTTNPSLILAAAKQPTYAKLIDVAVEYGKKHGKTTEEQVENAVDRLLVEFGKEILKIVPGRVSTEVDARLSFDTQATIEKARHIIKLFEQEGVSKERVLIKIASTWEGIQAAKELEEKDGIHCNLTLLFSFVQAVACAEAQVTLISPFVGRILDWYKSSTGKDYKGEADPGVISVKKIYNYYKKYGYKTIVMGASFRSTDEIKNLAGVDYLTISPALLDKLMNSTEPFPRVLDPVSAKKEAGDKISYISDESKFRFDLNEDAMATEKLSEGIRKFSADIVTLFDLIEKKVTA

SEQ ID NO: 23 - DNA, 717 bp, Saccharomyces cerevisiae
  CDS (1)..(717)
atggtcaaaccaattatagctcccagtatccttgcttctgacttcgccaacttgggttgcgaatgtcataaggtcatcaacgccggcgcagattggttacatatcgatgtcatggacggccattttgttccaaacattactctgggccaaccaattgttacctccctacgtcgttctgtgccacgccctggcgatgctagcaacacagaaaagaagcccactgcgttcttcgattgtcacatgatggttgaaaatcctgaaaaatgggtcgacgattttgctaaatgtggtgctgaccaatttacgttccactacgaggccacacaagaccctttgcatttagttaagttgattaagtctaagggcatcaaagctgcatgcgccatcaaacctggtacttctgttgacgttttatttgaactagctcctcatttggatatggctcttgttatgactgtggaacctgggtttggaggccaaaaattcatggaagacatgatgccaaaagtggaaactttgagagccaagttcccccatttgaatatccaagtcgatggtggtttgggcaaggagaccatcccgaaagccgccaaagccggtgccaacgttattgtcgctggtaccagtgttttcactgcagctgacccgcacgatgttatctccttcatgaaagaagaagtctcgaaggaattgcgttctagagatttgctagattag

SEQ ID NO: 24 - protein, 238 aa, Saccharomyces cerevisiae
MVKPIIAPSILASDFANLGCECHKVINAGADWLHIDVMDGHFVPNITLGQPIVTSLRRSVPRPGDASNTEKKPTAFFDCHMMVENPEKWVDDFAKCGADQFTFHYEATQDPLHLVKLIKSKGIKAACAIKPGTSVDVLFELAPHLDMALVMTVEPGFGGQKFMEDMMPKVETLRAKFPHLNIQVDGGLGKETIPKAAKAGANVIVAGTSVFTAADPHDVISFMKEEVSKELRSRDLLD

SEQ ID NO: 25 - DNA, 777 bp, Saccharomyces cerevisiae
  CDS (1)..(777)
atggctgccggtgtcccaaaaattgatgcgttagaatctttgggcaatcctttggaggatgccaagagagctgcagcatacagagcagttgatgaaaatttaaaatttgatgatcacaaaattattggaattggtagtggtagcacagtggtttatgttgccgaaagaattggacaatatttgcatgaccctaaattttatgaagtagcgtctaaattcatttgcattccaacaggattccaatcaagaaacttgattttggataacaagttgcaattaggctccattgaacagtatcctcgcattgatatagcgtttgacggtgctgatgaagtggatgagaatttacaattaattaaaggtggtggtgcttgtctatttcaagaaaaattggttagtactagtgctaaaaccttcattgtcgttgctgattcaagaaaaaagtcaccaaaacatttaggtaagaactggaggcaaggtgttcccattgaaattgtaccttcctcatacgtgagggtcaagaatgatctattagaacaattgcatgctgaaaaagttgacatcagacaaggaggttctgctaaagcaggtcctgttgtaactgacaataataacttcattatcgatgcggatttcggtgaaatttccgatccaagaaaattgcatagagaaatcaaactgttagtgggcgtggtggaaacaggtttattcatcgacaacgcttcaaaagcctacttcggtaattctgacggtagtgttgaagttaccgaaaagtga

SEQ ID NO: 26 - protein, 258 aa, Saccharomyces cerevisiae
MAAGVPKIDALESLGNPLEDAKRAAAYRAVDENLKFDDHKIIGIGSGSTVVYVAERIGQYLHDPKFYEVASKFICIPTGFQSRNLILDNKLQLGSIEQYPRIDIAFDGADEVDENLQLIKGGGACLFQEKLVSTSAKTFIVVADSRKKSPKHLGKNWRQGVPIEIVPSSYVRVKNDLLEQLHAEKVDIRQGGSAKAGPVVTDNNNFIIDADFGEISDPRKLHREIKLLVGVVETGLFIDNASKAYFGNSDGSVEVTEK

SEQ ID NO: 27 - DNA, 21 bp, Artificial Sequence
  Synthetic oligonucleotide primer
tgggaatattaccgctcgaag

SEQ ID NO: 28 - DNA, 29 bp, Artificial Sequence
  Synthetic oligonucleotide primer
ctttaaaaaatttccaattttcctttacg

SEQ ID NO: 29 - DNA, 33 bp, Artificial Sequence
  Synthetic oligonucleotide primer
ggaaattttttaaagtcgcagccacgggtcaac

SEQ ID NO: 30 - DNA, 50 bp, Artificial Sequence
  Synthetic oligonucleotide primer
gtgaattgagtcattttttattattagtcttttttttttttgacaatatc

SEQ ID NO: 31 - DNA, 31 bp, Artificial Sequence
  Synthetic oligonucleotide primer
atgactcaattcactgacattgataagctag

SEQ ID NO: 32 - DNA, 37 bp, Artificial Sequence
  Synthetic oligonucleotide primer
ccttaaatcaacgtcatattctttattggctttatac

SEQ ID NO: 33 - DNA, 24 bp, Artificial Sequence
  Synthetic oligonucleotide primer
gacgttgatttaaggtggttccgg

SEQ ID NO: 34 - DNA, 25 bp, Artificial Sequence
  Synthetic oligonucleotide primer
atgtctgaaccagctcaaaagaaac

SEQ ID NO: 35 - DNA, 52 bp, Artificial Sequence
  Synthetic oligonucleotide primer
agctggttcagacattttgaatatgtattacttggttatggttatatatgac

SEQ ID NO: 36 - DNA, 25 bp, Artificial Sequence
  Synthetic oligonucleotide primer
actggtagagagcgactttgtatgc

SEQ ID NO: 37 - DNA, 41 bp, Artificial Sequence
  Synthetic oligonucleotide primer
caaagtcgctctctaccagtcgctttcaattcatttgggtg

SEQ ID NO: 38 - DNA, 26 bp, Artificial Sequence
  Synthetic oligonucleotide primer
tgtatatgagatagttgattgtatgc

SEQ ID NO: 39 - DNA, 39 bp, Artificial Sequence
  Synthetic oligonucleotide primer
actatctcatatacaatggtcaaaccaattatagctccc

SEQ ID NO: 40 - DNA, 25 bp, Artificial Sequence
  Synthetic oligonucleotide primer
aaatggatattgatctagatggcgg

SEQ ID NO: 41 - DNA, 41 bp, Artificial Sequence
  Synthetic oligonucleotide primer
gatcaatatccatttcttggtgtgtcatcggtagtaacgcc

SEQ ID NO: 42 - DNA, 35 bp, Artificial Sequence
  Synthetic oligonucleotide primer
agttttaattacaaaatggctgccggtgtcccaaa

SEQ ID NO: 43 - DNA, 36 bp, Artificial Sequence
  Synthetic oligonucleotide primer
ttgtaattaaaacttagattagattgctatgctttc

SEQ ID NO: 44 - DNA, 18 bp, Artificial Sequence
  Synthetic oligonucleotide primer
aggaacagccgtcaaggg

SEQ ID NO: 45 - DNA, 35 bp, Artificial Sequence
  Synthetic oligonucleotide primer
ttgacggctgttcctcttcccttttacagtgcttc

SEQ ID NO: 46 - DNA, 32 bp, Artificial Sequence
  Synthetic oligonucleotide primer
tttgttttgtgtgtaaatttagtgaagtactg

SEQ ID NO: 47 - DNA, 41 bp, Artificial Sequence
  Synthetic oligonucleotide primer
tacacacaaaacaaaatgtctcaaatttttaaggatatccc

SEQ ID NO: 48 - DNA, 39 bp, Artificial Sequence
  Synthetic oligonucleotide primer
agcgctcttactttagcgatcgcactagtttattgaaac

SEQ ID NO: 49 - DNA, 28 bp, Artificial Sequence
  Synthetic oligonucleotide primer
taaagtaagagcgctacattggtctacc

SEQ ID NO: 50 - DNA, 36 bp, Artificial Sequence
  Synthetic oligonucleotide primer
taacattcaacgctattactccgcaacgcttttctg

SEQ ID NO: 51 - DNA, 26 bp, Artificial Sequence
  Synthetic oligonucleotide primer
tagcgttgaatgttagcgtcaacaac

SEQ ID NO: 52 - DNA, 41 bp, Artificial Sequence
  Synthetic oligonucleotide primer
tttgtttgtttatgtgtgtttattcgaaactaagttcttgg

SEQ ID NO: 53 - DNA, 42 bp, Artificial Sequence
  Synthetic oligonucleotide primer
acataaacaaacaaaatgttgtgttcagtaattcagagacag

SEQ ID NO: 54 - DNA, 39 bp, Artificial Sequence
  Synthetic oligonucleotide primer
aaataatcggtgtcattagatgagagtcttttccagttc

SEQ ID NO: 55 - DNA, 25 bp, Artificial Sequence
  Synthetic oligonucleotide primer
tgacaccgattatttaaagctgcag

SEQ ID NO: 56 - DNA, 18 bp, Artificial Sequence
  Synthetic oligonucleotide primer
agagcgcgcctcgttcag

SEQ ID NO: 57 - DNA, 33 bp, Artificial Sequence
  Synthetic oligonucleotide primer
aacgaggcgcgctctaattccgctgtatagctc

SEQ ID NO: 58 - DNA, 45 bp, Artificial Sequence
  Synthetic oligonucleotide primer
ataatgtatgctatacgaagttatagggaaagatatgagctatac

SEQ ID NO: 59 - DNA, 43 bp, Artificial Sequence
  Synthetic oligonucleotide primer
tatagcatacattatacgaagttatacgacatcgtcgaatatg

SEQ ID NO: 60 - DNA, 33 bp, Artificial Sequence
  Synthetic oligonucleotide primer
tattaatttagtgtgtgtatttgtgtttgtgtg

SEQ ID NO: 61 - DNA, 34 bp, Artificial Sequence
  Synthetic oligonucleotide primer
cacactaaattaataatgagccatattcaacggg

SEQ ID NO: 62 - DNA, 38 bp, Artificial Sequence
  Synthetic oligonucleotide primer
tttagtagacatgcattacaaccaattaaccaattctg

SEQ ID NO: 63 - DNA, 37 bp, Artificial Sequence
  Synthetic oligonucleotide primer
tgcatgtctactaaactcacaaattagagcttcaatt

SEQ ID NO: 64 - DNA, 54 bp, Artificial Sequence
  Synthetic oligonucleotide primer
ataatgtatgctatacgaagttatgggtaataactgatataattaaattgaagc

SEQ ID NO: 65 - DNA, 47 bp, Artificial Sequence
  Synthetic oligonucleotide primer
tatagcatacattatacgaagttattgacaccgattatttaaagctg

SEQ ID NO: 66 - DNA, 39 bp, Artificial Sequence
  Synthetic oligonucleotide primer
attttactggctggagtatgctgcagctttaaataatcg

SEQ ID NO: 67 - DNA, 26 bp, Artificial Sequence
  Synthetic oligonucleotide primer
tccagccagtaaaatccatactcaac

SEQ ID NO: 68 - DNA, 20 bp, Artificial Sequence
  Synthetic oligonucleotide primer
gtctttttgccagccagtcc

SEQ ID NO: 69 - DNA, 33 bp, Artificial Sequence
  Synthetic oligonucleotide primer
cacaccttcccccttgatcctctagagtcgacc

SEQ ID NO: 70 - DNA, 35 bp, Artificial Sequence
  Synthetic oligonucleotide primer
gcggtaatattcccagatccccgggtaccgagctc

SEQ ID NO: 71 - DNA, 33 bp, Artificial Sequence
  Synthetic oligonucleotide primer
cggtacccggggatcctatgggacttccgggaa

SEQ ID NO: 72 - DNA, 46 bp, Artificial Sequence
  Synthetic oligonucleotide primer
taacattcaacgctatgtgtattacgatatagttaatagttgatag

SEQ ID NO: 73 - DNA, 26 bp, Artificial Sequence
  Synthetic oligonucleotide primer
tagcgttgaatgttagcgtcaacaac

SEQ ID NO: 74 - DNA, 41 bp, Artificial Sequence; identical to SEQ ID NO: 52
  Synthetic oligonucleotide primer

SEQ ID NO: 75 - DNA, 40 bp, Artificial Sequence
  Synthetic oligonucleotide primer
acataaacaaacaaaatgtctatcccagaaactcaaaaag

SEQ ID NO: 76 - DNA, 30 bp, Artificial Sequence
  Synthetic oligonucleotide primer
ttgtcctctgaggacataaaatacacaccg

SEQ ID NO: 77 - DNA, 34 bp, Artificial Sequence
  Synthetic oligonucleotide primer
gtcctcagaggacaattactccgcaacgcttttc

SEQ ID NO: 78 - DNA, 37 bp, Artificial Sequence
  Synthetic oligonucleotide primer
ggagaggccgcataataaagtaagagcgctacattgg

SEQ ID NO: 79 - DNA, 18 bp, Artificial Sequence
  Synthetic oligonucleotide primer
ttatgcggcctctcctgc

SEQ ID NO: 80 - DNA, 43 bp, Artificial Sequence
  Synthetic oligonucleotide primer
agactaataataaaaatgtcaaagagaaaagttgctattatcg

SEQ ID NO: 81 - DNA, 47 bp, Artificial Sequence
  Synthetic oligonucleotide primer
tttttattattagtcttttttttttttgacaatatctgtatgatttg

SEQ ID NO: 82 - DNA, 33 bp, Artificial Sequence
  Synthetic oligonucleotide primer
ggagattaccgaatctcgctcgcagccacgggt

SEQ ID NO: 83 - DNA, 21 bp, Artificial Sequence
  Synthetic oligonucleotide primer
gattcggtaatctccgagcag

SEQ ID NO: 84 - DNA, 41 bp, Artificial Sequence
  Synthetic oligonucleotide primer
acataagagatccgcgggtaataactgatataattaaattg

SEQ ID NO: 85 - DNA, 35 bp, Artificial Sequence
  Synthetic oligonucleotide primer
gcggatctcttatgtctttacgatttatagttttc

SEQ ID NO: 86 - DNA, 20 bp, Artificial Sequence
  Synthetic oligonucleotide primer
gagggttgggcattcatcag

SEQ ID NO: 87 - DNA, 32 bp, Artificial Sequence
  Synthetic oligonucleotide primer
aatgcccaaccctcgatcctctagagtcgacc

SEQ ID NO: 88 - DNA, 18 bp, Artificial Sequence
  Synthetic oligonucleotide primer
gatccccgggtaccgagc

SEQ ID NO: 89 - DNA, 37 bp, Artificial Sequence
  Synthetic oligonucleotide primer
ttttattattagtcttttttttttttgacaatatctg

SEQ ID NO: 90 - DNA, 28 bp, Artificial Sequence
  Synthetic oligonucleotide primer
taaagtaagagcgctacattggtctacc

SEQ ID NO: 91 - DNA, 40 bp, Artificial Sequence
  Synthetic oligonucleotide primer
agcgctcttactttattaagctgatttctttgctttcttc

SEQ ID NO: 92 - DNA, 37 bp, Artificial Sequence
  Synthetic oligonucleotide primer
agactaataataaaaatggcagttacgaacgttgcag

SEQ ID NO: 93 - DNA, 37 bp, Artificial Sequence; identical to SEQ ID NO: 89
  Synthetic oligonucleotide primer

SEQ ID NO: 94 - DNA, 28 bp, Artificial Sequence
  Synthetic oligonucleotide primer
taaagtaagagcgctacattggtctacc

SEQ ID NO: 95 - DNA, 39 bp, Artificial Sequence
  Synthetic oligonucleotide primer
agcgctcttactttactaaacaattctgaatgcatcgac

SEQ ID NO: 96 - DNA, 40 bp, Artificial Sequence
  Synthetic oligonucleotide primer
agactaataataaaaatgaaccaacaagacatagaacaag

SEQ ID NO: 97 - DNA, 26 bp, Artificial Sequence
  Synthetic oligonucleotide primer
caatatctgctgtacaagcatacacc

SEQ ID NO: 98 - DNA, 48 bp, Artificial Sequence
  Synthetic oligonucleotide primer
ctatacagcggaattttatggttatttaactgtaacgaataggctagc

SEQ ID NO: 99 - DNA, 27 bp, Artificial Sequence
  Synthetic oligonucleotide primer
aattccgctgtatagctcatatctttc

SEQ ID NO: 100 - DNA, 25 bp, Artificial Sequence
  Synthetic oligonucleotide primer
gtatgctgcagctttaaataatcgg

SEQ ID NO: 101 - DNA, 45 bp, Artificial Sequence
  Synthetic oligonucleotide primer
aaagctgcagcatacccttatatctatgcagttggttgtgaaatc

SEQ ID NO: 102 - DNA, 17 bp, Artificial Sequence
  Synthetic oligonucleotide primer
gaaggaacagctgggcc

SEQ ID NO: 103 - DNA, 33 bp, Artificial Sequence
  Synthetic oligonucleotide primer
cccagctgttccttcgatcctctagagtcgacc

SEQ ID NO: 104 - DNA, 33 bp, Artificial Sequence
  Synthetic oligonucleotide primer
gtacagcagatattggatccccgggtaccgagc

SEQ ID NO: 105 - DNA, 26 bp, Artificial Sequence
  Synthetic oligonucleotide primer
caatatctgctgtacaagcatacacc

SEQ ID NO: 106 - DNA, 42 bp, Artificial Sequence
  Synthetic oligonucleotide primer
aagaagaaaactagtctatagtccatagaggtttctttcttg

SEQ ID NO: 107 - DNA, 30 bp, Artificial Sequence
  Synthetic oligonucleotide primer
actagttttcttctttcctcctcttctttg

SEQ ID NO: 108 - DNA, 21 bp, Artificial Sequence
  Synthetic oligonucleotide primer
cgttggttgaaacgttgtctg

SEQ ID NO: 109 - DNA, 38 bp, Artificial Sequence
  Synthetic oligonucleotide primer
acgtttcaaccaacgaattccgctgtatagctcatatc

SEQ ID NO: 110 - DNA, 25 bp, Artificial Sequence
  Synthetic oligonucleotide primer
gtatgctgcagctttaaataatcgg

SEQ ID NO: 111 - DNA, 45 bp, Artificial Sequence; identical to SEQ ID NO: 101
  Synthetic oligonucleotide primer

SEQ ID NO: 112 - DNA, 17 bp, Artificial Sequence
  Synthetic oligonucleotide primer
gaaggaacagctgggcc

SEQ ID NO: 113 - DNA, 33 bp, Artificial Sequence; identical to SEQ ID NO: 103
  Synthetic oligonucleotide primer

SEQ ID NO: 114 - DNA, 33 bp, Artificial Sequence; identical to SEQ ID NO: 104
  Synthetic oligonucleotide primer

SEQ ID NO: 115 - DNA, 20 bp, Artificial Sequence
  Synthetic oligonucleotide primer
aggctactgcgccaattgat

SEQ ID NO: 116 - DNA, 20 bp, Artificial Sequence
  Synthetic oligonucleotide primer
tgccctacacgttcgctatg

SEQ ID NO: 117 - DNA, 29 bp, Artificial Sequence
  Synthetic oligonucleotide primer
ggttttttctccttgacgttaaagtatag

SEQ ID NO: 118 - DNA, 26 bp, Artificial Sequence
  Synthetic oligonucleotide primer
ttagttatgtcacgcttacattcacg

SEQ ID NO: 119 - DNA, 22 bp, Artificial Sequence
  Synthetic oligonucleotide primer
atgtctaacttgttgactgttc

SEQ ID NO: 120 - DNA, 23 bp, Artificial Sequence
  Synthetic oligonucleotide primer
tcaatcaccatcttccaacaatc

(19) United States
(12) Patent Application Publication
(10) Pub. No.: US 2019/0211366 A1
(43) Pub. Date: Jul. 11, 2019

(54) RECOMBINANT YEAST AND A METHOD FOR PRODUCING ETHANOL USING THE SAME

(71) Applicant: TOYOTA JIDOSHA KABUSHIKI KAISHA, Toyota-shi, Aichi (JP)

(72) Inventors: Toru ONISHI, Toyota-shi (JP); Nobuki TADA, Nisshin-shi (JP)

(73) Assignee: Toyota Jidosha Kabushiki Kaisha, Toyota-shi, Aichi (JP)

(21) Appl. No.: 16/312,380

(22) PCT Filed: May 8, 2017
(86) PCT No.: PCT/JP2017/017331

§ 371 Date: Dec. 21, 2018

(30) Foreign Application Priority Data
Jun. 24, 2016 (JP) 2016-I26016

(51) Int. Cl. C12P 7/10; C12N 9/24; C12N 9/02; C12N 15/81; C12N 9/90

(52) U.S. Cl. CPC C12P 7/10; C12N 9/2402; C12N 9/0008; C12Y 503/01005; C12N 9/90; C12Y 102/0101; C12N 15/81

(57) ABSTRACT

The invention is intended to metabolize acetic acid in a medium at the time of culture, such as ethanol fermentation by yeast, and to reduce acetic acid concentration. Specifically, the invention relates to a recombinant yeast resulting from introduction of the acetaldehyde dehydrogenase gene (EC 1.2.1.10) and regulation of an enzyme involved with trehalose accumulation.

### TECHNICAL FIELD

The present invention relates to a recombinant yeast and a method for producing ethanol using the same.

### BACKGROUND ART

A cellulosic biomass is an effective starting material for a useful alcohol, such as ethanol, or an organic acid. In order to increase the amount of ethanol produced with the use of a cellulosic biomass, yeast strains capable of utilizing a xylose, which is a pentose, as a substrate have been developed. For example, Patent Literature 1 discloses a recombinant yeast strain resulting from incorporation of a xylose reductase gene and a xylitol dehydrogenase gene derived from Pichia stipitis and a xylulokinase gene derived from S. cerevisiae into its chromosome.

It is known that a large amount of acetic acid is contained in a hydrolysate of a cellulosic biomass and that acetic acid inhibits ethanol fermentation by a yeast strain. In the case of a yeast strain into which a xylose-assimilating gene has been introduced, in particular, acetic acid is known to inhibit ethanol fermentation carried out with the use of xylose as a saccharide source at a significant level (Non-Patent Literature 1 and 2).

A mash (moromi) resulting from fermentation of a cellulosic biomass saccharified with a cellulase is mainly composed of unfermented residue, poorly fermentable residue, enzymes, and fermenting microorganisms. Use of a mash-containing reaction solution for the subsequent fermentation process enables the reuse of fermenting microorganisms, reduction of the quantity of fermenting microorganisms to be introduced, and cost reduction. In such a case, however, acetic acid contained in the mash is simultaneously introduced, the concentration of acetic acid contained in a fermentation medium is increased as a consequence, and this may inhibit ethanol fermentation. In the case of a continuous fermentation technique in which the mash in a fermentation tank is transferred to a flash tank in which a reduced pressure level is maintained, ethanol is removed from the flash tank, and the mash is returned to the fermentation tank, although removal of acetic acid from the mash is difficult. Thus, inhibition of acetic acid-mediated fermentation would be critical.

In order to avoid inhibition of fermentation by acetic acid, there are reports concerning ethanol fermentation ability in the presence of acetic acid that has been improved by means of LPP1 or ENA1 gene overexpression (Non-Patent Literature 3) or FPS1 gene disruption (Non-Patent Literature 4) of Saccharomyces cerevisiae, which is a strain generally used for ethanol fermentation. However, such literature reports the results concerning ethanol fermentation conducted with the use of a glucose substrate, and the effects on ethanol fermentation conducted with the use of a xylose substrate, which is inhibited by acetic acid at a significant level, remain unknown. Even if the mutant yeast strains reported in such literature were used, the amount of acetic acid carry-over, which would be problematic at the time of the reuse of fermenting microorganisms or continuous fermentation, would not be reduced.

Alternatively, inhibition of fermentation by acetic acid may be avoided by metabolization of acetic acid in a medium simultaneously with ethanol fermentation. However, acetic acid metabolism is an aerobic reaction, which overlaps the metabolic pathway of ethanol. While acetic acid metabolism may be achieved by conducting fermentation under aerobic conditions, accordingly, ethanol as a target substance would also be metabolized.

As a means for metabolizing acetic acid under anaerobic conditions in which ethanol is not metabolized, assimilation of acetic acid achieved by introduction of the gene encoding acetaldehyde dehydrogenase (EC 1.2.1.10) into a Saccharomyces cerevisiae strain in which the GPD1 and GPD2 genes of the pathway of glycerin production had been destroyed has been reported (Non-Patent Literatures 5 and 6 and Patent Literatures 2 to 4). Acetaldehyde dehydrogenase catalyzes the reversible reaction described below.

Acetaldehyde+NAD++coenzyme A⇔acetyl coenzyme A+NADH+H⁺

The pathway of glycerin production mediated by the GPD1 and GPD2 genes is a pathway that oxidizes excessive coenzyme NADH resulting from metabolism into NAD⁺, as shown in the following chemical reaction.

0.5 glucose+NADH+H⁺+ATP→glycerin+NAD⁺+ADP+Pi

The reaction pathway is destructed by disrupting the GPD1 and GPD2 genes, excessive coenzyme NADH is supplied through introduction of acetaldehyde dehydrogenase, and the reaction proceeds as shown below.

Acetyl coenzyme A+NADH+H⁺→acetaldehyde+NAD⁺+coenzyme A

Acetyl coenzyme A is synthesized from acetic acid by acetyl-CoA synthetase, and acetaldehyde is converted into ethanol. Eventually, excessive coenzyme NADH is oxidized and acetic acid is metabolized, as shown in the following chemical reaction.

Acetic acid+2NADH+2H⁺+ATP→ethanol+NAD++AMP+Pi

As described above, it is necessary to destroy the glycerin pathway in order to impart acetic acid metabolizing ability to a yeast strain. However, the GPD1- and GPD2-disrupted strain is known to have significantly lowered fermentation ability, and utility at the industrial level is low.

There is a report concerning the supply of excess coenzyme NADH by causing an imbalance in the intracellular oxidation-reduction conditions depending on the difference of coenzyme dependence between the xylose reductase (XR) and the xylitol dehydrogenase (XDH) through introduction of XR and XDH of the xylose metabolic pathway instead of disruption of the GPD1 and GPD2 genes (Non-Patent Literature 7). Specifically, XR converts xylose into xylitol primarily using NADPH as a coenzyme to prepare NADP⁺ while XDH converts xylitol into xylulose using NAD⁺ as a coenzyme to prepare NADH. By causing an imbalance in coenzyme requirements of such enzymes, NADH is accumulated as a result. As a result of ethanol fermentation from xylose with the aid of a yeast strain into which XR and XDH have been introduced, however, an intermediate metabolite (i.e., xylitol) is accumulated. While acetic acid is metabolized, ethanol efficiency is poor, and such method is thus not practical.

A strain resulting from introduction of the acetaldehyde dehydrogenase into a strain that was not subjected to GPD1 or GPD2 gene disruption has also been reported (Non-Patent Literature 8). While Non-Patent Literature 8 reports that the amount of acetic acid production is reduced upon introduction of the acetaldehyde dehydrogenase, it does not report that acetic acid in the medium would be reduced. In addition, Non-Patent Literature 8 does not relate to a xylose-assimilating yeast strain.

Also, there are reports concerning a xylose-assimilating yeast strain resulting from introduction of a xylose isomerase (XI) gene (derived from the intestinal protozoa of termites) (Patent Literature 5) and a strain resulting from further introduction of the acetaldehyde dehydrogenase gene (derived from Bifidobacterium adolescentis) into a xylose-assimilating yeast strain comprising a XI gene (derived from Piromyces sp. E2) introduced thereinto (Patent Literature 6), although the above literature does not report acetic acid assimilation at the time of xylose assimilation.

According to conventional techniques, as described above, there are no reports concerning techniques for efficiently metabolizing and degrading acetic acid under conditions in which neither the GPD1 nor GPD2 gene had been disrupted.

Meanwhile, it has been reported that NTH1 is a trehalose-degrading enzyme gene and disruption thereof would result in an increased amount of trehalose accumulated in a cell and freezing tolerance is exerted (Patent Literature 7). It has also been reported that ethanol tolerance and heat tolerance are enhanced (Patent Literature 8). However, there has been no report concerning an improvement in acetic acid assimilation as a result of trehalose accumulation at the time of ethanol fermentation.

### CITATION LIST

### Patent Literature

PTL 1: JP 2009-195220 A
PTL 2: WO 2011/010923
PTL 3: WO 2011/140386
PTL 4: WO 2014/074895
PTL 5: JP 2011-147445 A
PTL 6: JP 2010-239925 A
PTL 7: JP 1998-11777 A
PTL 8: JP 1998-243783 A

### Non Patent Literature

NPL 1: FEMS Yeast Research, vol. 9, 2009, pp. 358-364
NPL 2: Enzyme and Microbial Technology 33, 2003. pp. 786-792
NPL 3: Biotechnol. Bioeng., 2009, 103 (3): pp. 500-512
NPL 4: Biotechnol. Lett., 2011, 33: pp. 277-284
NPL 5: Appl. Environ. Microbiol., 2010, 76: pp. 190-195
NPL 6: Appl. Environ. Microbiol., 2015, 81 81: 08-17
NPL 7: Nat Commun., 2013, 4, 2580
NPL 8: Biotechnol. Lett. 2011, 33: 1375-1380

### SUMMARY OF INVENTION

### Technical Problem

As described above, ethanol fermentation with the aid of a yeast involves disruption of the glycerin production pathway in combination with introduction of acetaldehyde dehydrogenase. Thus, acetic acid can be assimilated to a certain extent, although the ethanol fermentation ability of such yeast is deteriorated. In the case of a yeast involving only the introduction of acetaldehyde dehydrogenase, however, acetic acid assimilation ability of such yeast is insufficient.

Under the above circumstances, it is an object of the present invention to provide a recombinant yeast having improved acetic acid assimilation ability without involving the disruption of the glycerin production pathway, which would deteriorate the ethanol fermentation ability, and a method for producing ethanol using such recombinant yeast.

### Solution to Problem

The present inventors have conducted concentrated studies in order to attain the above objects. As a result, they have found that a recombinant yeast obtained by regulating the trehalose production pathway and increasing the amount of trehalose accumulated in a cell, as well as introducing the acetaldehyde dehydrogenase gene, would be capable of enhancing the acetic acid assimilation ability at the time of culture such as ethanol fermentation. This has led to the completion of the present invention.

The present invention includes the following.

(1) A recombinant yeast resulting from the introduction of the acetaldehyde dehydrogenase gene (EC 1.2.1.10) and the regulation of an enzyme involved with trehalose accumulation.

(2) The recombinant yeast according to (1), wherein the acetaldehyde dehydrogenase gene encodes the acetaldehyde dehydrogenase derived from E. coli.

(3) The recombinant yeast according to (2), wherein the acetaldehyde dehydrogenase derived from E. coli is the protein (a) or (b) below:

(a) a protein consisting of the amino acid sequence as shown in SEQ ID NO: 2, 4, or 6; or

(b) a protein consisting of an amino acid sequence having 90% or more identity with the amino acid sequence as shown in SEQ ID NO: 2, 4, or 6 and having acetaldehyde dehydrogenase activity.

(4) The recombinant yeast according to any one of (1) to (3), wherein the regulation of an enzyme involved with trehalose accumulation is a lowered expression level of the trehalase gene.

(5) The recombinant yeast according to any one of (1) to (4), which further comprises the xylose isomerase gene introduced thereinto.

(6) The recombinant yeast according to (5), wherein the xylose isomerase is the protein (a) or (b) below:

(a) a protein consisting of the amino acid sequence as shown in SEQ ID NO: 12; or

(b) a protein consisting of an amino acid sequence having 90% or more identity with the amino acid sequence as shown in SEQ ID NO: 12 and having enzyme activity of converting xylose into xylulose.

(7) The recombinant yeast according to any one of (1) to (6), which further comprises the xylulokinase gene introduced thereinto.

(8) The recombinant yeast according to any one of (1) to (7), which further comprises a gene encoding an enzyme selected from the group of enzymes constituting a non-oxidative process pathway in the pentose phosphate pathway introduced thereinto.

(9) The recombinant yeast according to (8), wherein the group of enzymes constituting a non-oxidative process pathway in the pentose phosphate pathway includes ribose-5-phosphate isomerase, ribulose-5-phosphate-3-epimerase, transketolase, and transaldolase.

(10) The recombinant yeast according to any one of (1) to (9), wherein the alcohol dehydrogenase gene having activity of converting acetaldehyde into ethanol is expressed at a high level.

(11) The recombinant yeast according to any one of (1) to (10), wherein the expression level of the alcohol dehydrogenase gene having activity of converting ethanol into acetaldehyde is lowered.

(12) A method for producing ethanol comprising a step of ethanol fermentation via culture of the recombinant yeast according to any one of (1) to (11) in a medium containing glucose and/or xylose.

(13) The method according to (12), wherein the medium contains cellulose and the ethanol fermentation proceeds simultaneously with saccharification of at least the cellulose.

This Description includes part or all of the contents as disclosed in the Description of Japanese Patent Application No. 2016-126016, to which the present application claims priority.

### Advantageous Effects of Invention

With the use of the recombinant yeast according to the present invention, acetic acid concentration in a medium can be lowered at the time of culture, such as ethanol fermentation, and fermentation inhibited by acetic acid can be effectively avoided. As a result, the method for ethanol production of the present invention enables maintenance of ethanol fermentation efficiency at a high level with the use of the recombinant yeast according to the present invention, thereby achieving an excellent ethanol yield.

Accordingly, the method for ethanol production according to the present invention enables reduction of the amount of acetic acid carry-over at the time of, for example, the reuse of the recombinant yeast or use thereof for continuous culture, thereby allowing maintenance of an excellent ethanol yield.

### DESCRIPTION OF EMBODIMENTS

[Recombinant Yeast]

The recombinant yeast according to the present invention is obtained via introduction of the acetaldehyde dehydrogenase gene (EC 1.2.1.10) thereinto and regulation of an enzyme involved with trehalose accumulation so as to increase the amount of trehalose accumulated in a cell. The recombinant yeast according to the present invention is characterized in that it is capable of metabolizing acetic acid contained in a medium and lowering the acetic acid concentration in a medium upon culture such as ethanol fermentation.

An acetaldehyde dehydrogenase gene to be introduced into a yeast is not particularly limited, and a gene derived from any species of organism may be used. When acetaldehyde dehydrogenase genes derived from organisms other than a fungus such as yeast (e.g., genes derived from bacteria, animals, plants, insects, or algae) are used, it is preferable that the nucleotide sequence of the gene be modified in accordance with the frequency of codon usage in a yeast into which the gene of interest is to be introduced.

More specifically, the mhpF gene of E. coli and the ALDH 1 gene of Entamoeba histolytica as disclosed in Applied and Environmental Microbiology, May 2004, pp. 2892-2897, Vol. 70, No. 5 can be used as the acetaldehyde dehydrogenase genes. The nucleotide sequence of the mhpF gene of E. coli and the amino acid sequence of a protein encoded by the mhpF gene are shown in SEQ ID NOs: 1 and 2, respectively.

The acetaldehyde dehydrogenase gene is not limited to the gene identified by SEQ ID NOs: 1 and 2. It may be a paralogous gene or a homologous gene in the narrow sense having different nucleotide and amino acid sequences, provided that it is an enzyme defined with EC 1.2.1.10. Examples of the acetaldehyde dehydrogenase genes include an adhE gene and an eutE gene of E. coli, an acetaldehyde dehydrogenase gene derived from Clostridium beijerinckii, and an acetaldehyde dehydrogenase gene derived from Chlamydomonas reinhardtii. Here, the nucleotide sequence of the adhE gene of E. coli and the amino acid sequence of a protein encoded by the adhE gene are shown in SEQ ID NOs: 3 and 4, respectively. The nucleotide sequence of the eutE gene of E. coli and the amino acid sequence of a protein encoded by the eutE gene are shown in SEQ ID NOs: 5 and 6, respectively. In addition, the nucleotide sequence of the acetaldehyde dehydrogenase gene derived from Clostridium beijerinckii and the amino acid sequence of a protein encoded by the gene are shown in SEQ ID NOs: 7 and 8, respectively. Further, the nucleotide sequence of the acetaldehyde dehydrogenase gene derived from Chlamydomonas reinhardtii and the amino acid sequence of a protein encoded by the gene are shown in SEQ ID NOs: 9 and 10, respectively.

The acetaldehyde dehydrogenase genes are not limited to the genes identified by SEQ ID NOs: 1 and 2, 3 and 4, 5 and 6, 7 and 8, and 9 and 10. For example, it may be a gene encoding a protein consisting of an amino acid sequence having 70% or more, preferably 80% or more, more preferably 90% or more, and most preferably 95% or more sequence similarity to or identity with the amino acid sequence as shown in SEQ ID NO: 2, 4, 6, 8, or 10 and having acetaldehyde dehydrogenase activity. The degree of sequence similarity or identity can be determined using the BLASTN or BLASTX Program equipped with the BLAST algorithm (at default settings). The degree of sequence similarity is determined by subjecting a pair of amino acid sequences to pairwise alignment analysis, identifying completely identical amino acid residues and amino acid residues exhibiting physicochemically similar functions, determining the total number of such amino acid residues, and calculating the percentage of all the amino acid residues subjected to comparison accounted for by the total number of the aforementioned amino acid residues. The degree of sequence identity is determined by subjecting a pair of amino acid sequences to pairwise alignment analysis, identifying completely identical amino acid residues, and calculating the percentage of all the amino acid residues subjected to comparison accounted for by such completely identical amino acid residues.

Further, the acetaldehyde dehydrogenase genes are not limited to the genes identified by SEQ ID NOs: 1 and 2, 3 and 4, 5 and 6, 7 and 8, and 9 and 10. For example, it may be a gene encoding a protein comprising an amino acid sequence derived from the amino acid sequence as shown in SEQ ID NO: 2, 4, 6, 8, or 10 by substitution, deletion, insertion, or addition of one or several amino acids and having acetaldehyde dehydrogenase activity. The term “several” used herein refers to, for example, 2 to 30, preferably 2 to 20, more preferably 2 to 10, and most preferably 2 to 5.

Furthermore, the acetaldehyde dehydrogenase genes are not limited to the genes identified by SEQ ID NOs: 1 and 2, 3 and 4, 5 and 6, 7 and 8, and 9 and 10. For example, it may be a gene hybridizing under stringent conditions to the full-length sequence or a partial sequence of a complementary strand of DNA consisting of the nucleotide sequence as shown in SEQ ID NO: 1, 3, 5, 7, or 9 and encoding a protein having acetaldehyde dehydrogenase activity. Under “stringent conditions,” so-called specific hybrids are formed, but non-specific hybrids are not formed. Such conditions can be adequately determined with reference to, for example. Molecular Cloning: A Laboratory Manual (Third Edition). Specifically, the degree of stringency can be determined in accordance with the temperature and the salt concentration of a solution used for Southern hybridization and the temperature and the salt concentration of a solution used for the step of washing in Southern hybridization. Under stringent conditions, more specifically, the sodium concentration is 25 to 500 mM and preferably 25 to 300 mM, and the temperature is 42 degrees C. to 68 degrees C. and preferably 42 degrees C. to 65 degrees C., for example. Further specifically, the sodium concentration is 5×SSC (83 mM NaCl, 83 mM sodium citrate), and the temperature is 42 degrees C.

As described above, whether or not a gene consisting of a nucleotide sequence that differs from the sequence as shown in SEQ ID NO: 1, 3, 5, 7, or 9 or a gene encoding an amino acid sequence that differs from the sequence as shown in SEQ ID NO: 2, 4, 6, 8, or 10 would function as an acetaldehyde dehydrogenase gene may be determined by, for example, preparing an expression vector comprising the gene of interest incorporated into an adequate site between an appropriate promoter and an appropriate terminator, transforming a host such as E. coli using such expression vector, and assaying acetaldehyde dehydrogenase activity of the protein expressed. Acetaldehyde dehydrogenase activity can be assayed by preparing a solution containing acetaldehyde, CoA, and NAD⁺ as substrates, allowing the target protein to react at adequate temperature, and converting the generated acetyl phosphate into acetyl phosphate with the aid of a phosphate acetyl transferase or spectroscopically assaying the generated NADH.

The recombinant yeast according to the present invention is obtained by regulation of an enzyme involved with trehalose accumulation, so as to increase the amount of trehalose accumulated in a cell. The term an “enzyme involved with trehalose accumulation” refers to an enzyme that directly or indirectly plays a role in trehalose production or degradation. Examples of enzymes involved with trehalose accumulation include trehalose-degrading enzyme (i.e., trehalase), such as neutral trehalase (NTH1) and acidic trehalase (ATH1). Regulation of an enzyme involved with trehalose accumulation allows a gene encoding such enzyme to be expressed at a high level so as to increase the amount of trehalose accumulated in a cell or the expression level of the gene encoding the enzyme to decrease. High-level gene expression is achieved by, for example, a method in which a relevant foreign gene is introduced into a yeast or a method in which a relevant endogenous gene promoter is substituted with a promoter for high-level expression. In order to lower the expression level of a gene such as a trehalase gene, a promoter or terminator of the endogenous gene may be modified or the gene may be disrupted. Such gene may be disrupted by disrupting one or both of the alleles existing in a diploid yeast. Examples of techniques for suppressing gene expression include the transposon technique, the transgene technique, the post-transcriptional gene silencing technique, the RNAi technique, the nonsense mediated decay (NMD) technique, the ribozyme technique, the anti-sense technique, the miRNA (micro-RNA) technique, and the siRNA (small interfering RNA) technique.

The recombinant yeast according to the present invention may have xylose-metabolizing ability as a result of introduction of a xylose isomerase gene thereinto. That is, the term “yeast having xylose-metabolizing ability” refers to any of a yeast imparted with xylose-metabolizing ability as a result of introduction of a xylose isomerase gene into a yeast that does not inherently have xylose-metabolizing ability, a yeast imparted with xylose-metabolizing ability as a result of introduction of a xylose isomerase gene and another xylose metabolism-associated gene into a yeast that does not inherently have xylose-metabolizing ability, or a yeast that inherently has xylose-metabolizing ability.

A yeast having xylose-metabolizing ability is capable of assimilating xylose contained in a medium to produce ethanol. Xylose contained in a medium may be obtained by saccharification of xylan or hemicellulose comprising xylose as a constituent sugar. Alternatively, it may be a substance supplied to a medium as a result of saccharification of xylan or hemicellulose contained in a medium by a carbohydrase. The latter case refers to the so-called simultaneous saccharification and fermentation system.

Xylose isomerase (XI) genes are not particularly limited, and genes derived from any organism species may be used. For example, a plurality of the xylose isomerase genes derived from the intestinal protozoa of termites disclosed in JP 2011-147445 A can be used without particular limitation. Examples of the xylose isomerase genes that can be used include a gene derived from the anaerobic fungus Piromyces sp. strain E2 (JP 2005-514951 A), a gene derived from the anaerobic fungus Cyllamyces aberensis, a gene derived from a bacterial strain (i.e., Bacteroides thetaiotaomicron), a gene derived from a bacterial strain (i.e., Clostridium phytofermentans), and a gene derived from a strain of the Streptomyces murinus cluster.

Specifically, use of a xylose isomerase gene derived from the intestinal protozoa of Reticulitermes speratus as the xylose isomerase gene is preferable. The nucleotide sequence of the coding region of the xylose isomerase gene derived from the intestinal protozoa of Reticulitermes speratus and the amino acid sequence of a protein encoded by such gene are shown in SEQ ID NOs: 11 and 12, respectively.

The xylose isomerase gene is not limited to the gene identified by SEQ ID NOs: 11 and 12. It may be a paralogous gene or a homologous gene in the narrow sense having different nucleotide and amino acid sequences.

The xylose isomerase gene is not limited to the gene identified by SEQ ID NOs: 11 and 12. For example, it may be a gene encoding a protein consisting of an amino acid sequence having 70% or more, preferably 80% or more, more preferably 90% or more, and most preferably 95% or more sequence similarity to or identity with the amino acid sequence as shown in SEQ ID NO: 12 and having xylose isomerase activity. The degree of sequence similarity or identity can be determined using the BLASTN or BLASTX Program equipped with the BLAST algorithm (at default settings). The degree of sequence similarity is determined by subjecting a pair of amino acid sequences to pairwise alignment analysis, identifying completely identical amino acid residues and amino acid residues exhibiting physicochemically similar functions, determining the total number of such amino acid residues, and calculating the percentage of all the amino acid residues subjected to comparison accounted for by the total number of such amino acid residues. The degree of sequence identity is determined by subjecting a pair of amino acid sequences to pairwise alignment analysis, identifying completely identical amino acid residues, and calculating the percentage of all the amino acid residues subjected to comparison accounted for by such amino acid residues.

Further, the xylose isomerase gene is not limited to the gene identified by SEQ ID NOs: 11 and 12. For example, it may be a gene encoding a protein comprising an amino acid sequence derived from the amino acid sequence as shown in SEQ ID NO: 12 by substitution, deletion, insertion, or addition of one or several amino acids and having xylose isomerase activity. The term “several” used herein refers to, for example, 2 to 30, preferably 2 to 20, more preferably 2 to 10, and most preferably 2 to 5.

Furthermore, the xylose isomerase gene is not limited to the gene identified by SEQ ID NOs: 11 and 12. For example, it may be a gene hybridizing under stringent conditions to the full-length sequence or a partial sequence of a complementary strand of DNA consisting of the nucleotide sequence as shown in SEQ ID NO: 11 and encoding a protein having xylose isomerase activity. Under “stringent conditions,” so-called specific hybrids are formed, but non-specific hybrids are not formed. Such conditions can be adequately determined with reference to, for example, Molecular Cloning: A Laboratory Manual (Third Edition). Specifically, the degree of stringency can be determined in accordance with the temperature and the salt concentration of a solution used for Southern hybridization and the temperature and the salt concentration of a solution used for the step of washing in Southern hybridization. Under stringent conditions, more specifically, the sodium concentration is 25 to 500 mM and preferably 25 to 300 mM, and the temperature is 42 degrees C. to 68 degrees C. and preferably 42 degrees C. to 65 degrees C., for example. Further specifically, the sodium concentration is 5×SSC (83 mM NaCl, 83 mM sodium citrate), and the temperature is 42 degrees C.

As described above, whether or not a gene consisting of a nucleotide sequence that differs from the sequence as shown in SEQ ID NO: 11 or a gene encoding an amino acid sequence that differs from the sequence as shown in SEQ ID NO: 12 would function as the xylose isomerase gene may be determined by, for example, preparing an expression vector comprising the gene of interest incorporated into an adequate site between an appropriate promoter and an appropriate terminator, transforming a host such as E. coli using such expression vector, and assaying xylose isomerase activity of the protein expressed. The term “xylose isomerase activity” refers to activity of isomerizing xylose into xylulose. Thus, xylose isomerase activity can be evaluated by preparing a solution containing xylose as a substrate, allowing the target protein to react at an adequate temperature, and measuring the amount of xylose that had decreased and/or the amount of xylulose that had been produced.

Use of a gene encoding a mutant xylose isomerase consisting of an amino acid sequence derived from the amino acid sequence as shown in SEQ ID NO: 12 via introduction of a particular mutation into a particular amino acid residue and having improved xylose isomerase activity is particularly preferable as a xylose isomerase gene. A specific example of a gene encoding a mutant xylose isomerase is a gene encoding an amino acid sequence derived from the amino acid sequence as shown in SEQ ID NO: 12 by substitution of asparagine at position 337 with cysteine. Xylose isomerase activity of the xylose isomerase consisting of an amino acid sequence derived from the amino acid sequence as shown in SEQ ID NO: 12 by substitution of asparagine at position 337 with cysteine is superior to that of a wild-type xylose isomerase. A mutant xylose isomerase is not limited to one resulting from substitution of asparagine at position 337 with cysteine. A mutant xylose isomerase may result from substitution of asparagine at position 337 with amino acid other than cysteine, substitution of another amino acid residue in addition to asparagine at position 337 with another amino acid, or substitution of another amino acid residue other than that at position 337.

Examples of xylose metabolism-associated genes other than the xylose isomerase genes include a xylose reductase gene encoding a xylose reductase that converts xylose into xylitol, a xylitol dehydrogenase gene encoding a xylitol dehydrogenase that converts xylitol into xylulose, and a xylulokinase gene encoding a xylulokinase that phosphorylates xylulose to produce xylulose 5-phosphate. Xylulose 5-phosphate produced by a xylulokinase will be metabolized in a pentose phosphate pathway.

Examples of xylose metabolism-associated genes include, but are not particularly limited to, a xylose reductase gene and a xylitol dehydrogenase gene derived from Pichia stipitis and a xylulokinase gene derived from Saccharomyces cerevisiae (see Eliasson A. et al., Appl. Environ. Microbiol., 66: 3381-3386; and Toivari M. N. et al., Metab. Eng., 3: 236-249). In addition, xylose reductase genes derived from Candida tropicalis and Candida prapsilosis, xylitol dehydrogenase genes derived from Candida tropicalis and Candida prapsilosis, and a xylulokinase gene derived from Pichia stipitis can be used.

Examples of yeasts that inherently have xylose-metabolizing ability include, but are not particularly limited to, Pichia stipitis, Candida tropicalis, and Candida prapsilosis.

The recombinant yeast according to the present invention may further comprise other gene(s) introduced thereinto, and such other gene(s) are not particularly limited. For example, a gene involved in the metabolism of sugar such as glucose may be introduced into such recombinant yeast. For example, a recombinant yeast can have beta-glucosidase activity resulting from the introduction of the beta-glucosidase gene.

The term “beta-glucosidase activity” used herein refers to the activity of catalyzing a hydrolysis reaction of a beta-glycoside bond of a sugar. Specifically, beta-glucosidase is capable of degrading a cellooligosaccharide, such as cellobiose, into glucose. The beta-glucosidase gene can be introduced in the form of a cell-surface display gene. The term “cell-surface display gene” used herein refers to a gene that is modified to display a protein to be encoded by the gene on a cell surface. For example, a cell-surface display beta-glucosidase gene is a gene resulting from fusion of a beta-glucosidase gene with a cell-surface localized protein gene. A cell-surface localized protein is fixed and present on a yeast cell surface layer. Examples include agglutinative proteins, such as alpha- or a-agglutinin, and FLO proteins. In general, a cell-surface localized protein comprises an N-terminal secretory signal sequence and a C-terminal GPI anchor attachment recognition signal. While a cell-surface localized protein shares properties with a secretory protein in terms of the presence of a secretory signal, the cell-surface localized protein differs from the secretory protein in that the cell-surface localized protein is transported while fixed to a cell membrane through a GPI anchor. When a cell-surface localized protein passes through a cell membrane, a GPI anchor attachment recognition signal sequence is selectively cut, it binds to a GPI anchor at a newly protruded C-terminal region, and it is then fixed to the cell membrane. Thereafter, the root of the GPI anchor is cut by phosphatidylinositol-dependent phospholipase C (PI-PLC). Subsequently, a protein separated from the cell membrane is integrated into a cell wall, fixed onto a cell surface layer, and then localized on a cell surface layer (see, for example, JP 2006-174767 A).

The beta-glucosidase gene is not particularly limited, and an example is a beta-glucosidase gene derived from Aspergillus aculeatus (Murai, et al., Appl. Environ. Microbiol., 64: 4857-4861). In addition, a beta-glucosidase gene derived from Aspergillus oryzae, a beta-glucosidase gene derived from Clostridium cellulovorans, and a beta-glucosidase gene derived from Saccharomycopsis fibligera can be used.

In addition to or other than the beta-glucosidase gene, a gene encoding another cellulase-constituting enzyme may have been introduced into the recombinant yeast according to the present invention. Examples of cellulase-constituting enzymes other than beta-glucosidase include exo-cellobiohydrolases that liberate cellobiose from the terminus of crystalline cellulose (CBH1 and CBH2) and endo-glucanase (EG) that cannot degrade crystalline cellulose but cleaves a non-crystalline cellulose (amorphous cellulose) chain at random.

Examples of other genes to be introduced into a recombinant yeast include an alcohol dehydrogenase gene (the ADH1 gene) having activity of converting acetaldehyde into ethanol, an acetyl-CoA synthetase gene (the ACS 1 gene) having activity of converting acetic acid into acetyl-CoA, and genes having activity of converting acetaldehyde into acetic acid (i.e., the ALD4, ALD5, and ALD6 genes). The alcohol dehydrogenase gene (the ADH2 gene) having activity of converting ethanol into acetaldehyde may be disrupted.

In addition, it is preferable that the recombinant yeast according to the present invention allow high-level expression of the alcohol dehydrogenase gene (the ADH1 gene) having activity of converting acetaldehyde into ethanol. In order to realize high-level expression of such gene, for example, a promoter of the inherent gene may be replaced with a promoter intended for high-level expression, or an expression vector enabling expression of such gene may be introduced into a yeast.

The nucleotide sequence of the ADH1 gene of Saccharomyces cerevisiae and the amino acid sequence of a protein encoded by such gene are shown in SEQ ID NOs: 13 and 14, respectively. The alcohol dehydrogenase gene to be expressed at high level is not limited to the gene identified by SEQ ID NOs: 13 and 14. It may be a paralogous gene or a homologous gene in the narrow sense having different nucleotide and amino acid sequences.

The alcohol dehydrogenase gene is not limited to the gene identified by SEQ ID NOs: 13 and 14. For example, it may be a gene encoding a protein consisting of an amino acid sequence having 70% or more, preferably 80% or more, more preferably 90% or more, and most preferably 95% or more sequence similarity to or identity with the amino acid sequence as shown in SEQ ID NO: 14 and having alcohol dehydrogenase activity. The degree of sequence similarity or identity can be determined using the BLASTN or BLASTX Program equipped with the BLAST algorithm (at default settings). The degree of sequence similarity is determined by subjecting a pair of amino acid sequences to pairwise alignment analysis, identifying completely identical amino acid residues and amino acid residues exhibiting physicochemically similar functions, determining the total number of such amino acid residues, and calculating the percentage of all the amino acid residues subjected to comparison accounted for by the total number of such amino acid residues. The degree of sequence identity is determined by subjecting a pair of amino acid sequences to pairwise alignment analysis, identifying completely identical amino acid residues, and calculating the percentage of all the amino acid residues subjected to comparison accounted for by the total number of such amino acid residues.

Further, the alcohol dehydrogenase gene is not limited to the gene identified by SEQ ID NOs: 13 and 14. For example, it may be a gene encoding a protein consisting of an amino acid sequence derived from the amino acid sequence as shown in SEQ ID NO: 14 by substitution, deletion, insertion, or addition of one or several amino acids and having alcohol dehydrogenase activity. The term “several” used herein refers to, for example, 2 to 30, preferably 2 to 20, more preferably 2 to 10, and most preferably 2 to 5.

Furthermore, the alcohol dehydrogenase gene is not limited to the gene identified by SEQ ID NOs: 13 and 14. For example, it may be a gene hybridizing under stringent conditions to the full-length sequence or a partial sequence of a complementary strand of DNA consisting of the nucleotide sequence as shown in SEQ ID NO: 13 and encoding a protein having alcohol dehydrogenase activity. Under “stringent conditions,” so-called specific hybrids are formed, but non-specific hybrids are not formed. Such conditions can be adequately determined with reference to, for example, Molecular Cloning: A Laboratory Manual (Third Edition). Specifically, the degree of stringency can be determined in accordance with the temperature and the salt concentration of a solution used for Southern hybridization and the temperature and the salt concentration of a solution used for the step of washing in Southern hybridization. Under stringent conditions, more specifically, the sodium concentration is 25 to 500 mM and preferably 25 to 300 mM, and the temperature is 42 degrees C. to 68 degrees C. and preferably 42 degrees C. to 65 degrees C., for example. Further specifically, the sodium concentration is 5×SSC (83 mM NaCl, 83 mM sodium citrate), and the temperature is 42 degrees C.

As described above, whether or not a gene consisting of a nucleotide sequence that differs from the sequence as shown in SEQ ID NO: 13 or a gene encoding an amino acid sequence that differs from the sequence as shown in SEQ ID NO: 14 would function as an alcohol dehydrogenase gene having activity of converting acetaldehyde into ethanol may be determined by, for example, preparing an expression vector comprising the gene of interest incorporated into an adequate site between an appropriate promoter and an appropriate terminator, transforming a host such as yeast using such expression vector, and assaying alcohol dehydrogenase activity of the protein expressed. Alcohol dehydrogenase activity of converting acetaldehyde into ethanol can be assayed by preparing a solution containing aldehyde and NADH or NADPH as substrates, allowing the target protein to react at adequate temperature, and assaying the generated alcohol or spectroscopically assaying NADT or NADP+.

The recombinant yeast according to the present invention is preferably characterized by a lowered expression level of the alcohol dehydrogenase gene (the ADH2 gene) having activity of converting ethanol into acetaldehyde. In order to lower the expression level of such gene, a promoter of the inherent gene of interest may be modified, or such gene may be deleted. In order to delete the gene, either or both of a pair of ADH2 genes present in a diploid recombinant yeast may be deleted. Examples of techniques for suppressing gene expression include the transposon technique, the transgene technique, post-transcriptional gene silencing, the RNAi technique, the nonsense mediated decay (NMD) technique, the ribozyme technique, the anti-sense technique, the miRNA (micro-RNA) technique, and the siRNA (small interfering RNA) technique.

The nucleotide sequence of the ADH2 gene of Saccharomyces cerevisiae and the amino acid sequence of a protein encoded by such gene are shown in SEQ ID NOs: 15 and 16, respectively. The target alcohol dehydrogenase gene is not limited to the gene identified by SEQ ID NOs: 15 and 16. It may be a paralogous gene or a homologous gene in the narrow sense having different nucleotide and amino acid sequences.

The alcohol dehydrogenase gene is not limited to the gene identified by SEQ ID NOs: 15 and 16. For example, it may be a gene encoding a protein comprising an amino acid sequence having 70% or more, preferably 80% or more, more preferably 90% or more, and most preferably 95% or more sequence similarity to or identity with the amino acid sequence as shown in SEQ ID NO: 16 and having alcohol dehydrogenase activity. The degree of sequence similarity or identity can be determined using the BLASTN or BLASTX Program equipped with the BLAST algorithm (at default settings). The degree of sequence similarity is determined by subjecting a pair of amino acid sequences to pairwise alignment analysis, identifying completely identical amino acid residues and amino acid residues exhibiting physicochemically similar functions, determining the total number of such amino acid residues, and calculating the percentage of all the amino acid residues subjected to comparison accounted for by the total number of such amino acid residues. The degree of sequence identity is determined by subjecting a pair of amino acid sequences to pairwise alignment analysis, identifying completely identical amino acid residues, and calculating the percentage of all the amino acid residues subjected to comparison accounted for by the total number of such amino acid residues.

Further, the alcohol dehydrogenase gene is not limited to the gene identified by SEQ ID NOs: 15 and 16. For example, it may be a gene encoding a protein consisting of an amino acid sequence derived from the amino acid sequence as shown in SEQ ID NO: 16 by substitution, deletion, insertion, or addition of one or several amino acids and having alcohol dehydrogenase activity. The term “several” used herein refers to, for example, 2 to 30, preferably 2 to 20, more preferably 2 to 10, and most preferably 2 to 5.

Furthermore, the alcohol dehydrogenase gene is not limited to the gene identified by SEQ ID NOs: 15 and 16. For example, it may be a gene hybridizing under stringent conditions to the full-length sequence or a partial sequence of a complementary strand of DNA consisting of the nucleotide sequence as shown in SEQ ID NO: 15 and encoding a protein having alcohol dehydrogenase activity. Under “stringent conditions,” so-called specific hybrids are formed, but non-specific hybrids are not formed. Such conditions can be adequately determined with reference to, for example. Molecular Cloning: A Laboratory Manual (Third Edition). Specifically, the degree of stringency can be determined in accordance with the temperature and the salt concentration of a solution used for Southern hybridization and the temperature and the salt concentration of a solution used for the step of washing in Southern hybridization. Under stringent conditions, more specifically, the sodium concentration is 25 to 500 mM and preferably 25 to 300 mM, and the temperature is 42 degrees C. to 68 degrees C. and preferably 42 degrees C. to 65 degrees C., for example. Further specifically, the sodium concentration is 5×SSC (83 mM NaCl, 83 mM sodium citrate), and the temperature is 42 degrees C.

As described above, whether or not a gene consisting of a nucleotide sequence that differs from the sequence as shown in SEQ ID NO: 15 or a gene encoding an amino acid sequence that differs from the sequence as shown in SEQ ID NO: 16 would function as an alcohol dehydrogenase gene having activity of converting ethanol into acetaldehyde may be determined by, for example, preparing an expression vector comprising the gene of interest incorporated into an adequate site between an appropriate promoter and an appropriate terminator, transforming a host such as yeast using such expression vector, and assaying alcohol dehydrogenase activity of the protein expressed. Alcohol dehydrogenase activity of converting ethanol into acetaldehyde can be assayed by preparing a solution containing alcohol and NAD⁺ or NADP⁺ as substrates, allowing the target protein to react at adequate temperature, and assaying the generated aldehyde or spectroscopically assaying NADH or NADPH.

Further examples of other genes that can be introduced into a recombinant yeast include genes associated with the metabolic pathway of L-arabinose, which is a pentose contained in hemicellulose constituting a biomass. Examples of such genes include an L-arabinose isomerase gene, an L-ribulokinase gene, and an L-ribulose-5-phosphate 4-epimerase gene derived from prokaryotes and an L-arabitol-4-dehydrogenase gene and an L-xylose reductase gene derived from eukaryotes.

In particular, an example of another gene to be introduced into a recombinant yeast is a gene capable of promoting the use of xylose in a medium. A specific example thereof is a gene encoding xylulokinase having activity of generating xylulose-5-phosphate using xylulose as a substrate. The metabolic flux of the pentose phosphate pathway can be improved through the introduction of the xylulokinase gene.

Further, a gene encoding an enzyme selected from the group of enzymes constituting a non-oxidative process pathway in the pentose phosphate pathway can be introduced into a recombinant yeast. Examples of enzymes constituting a non-oxidative process pathway in the pentose phosphate pathway include ribose-5-phosphate isomerase, ribulose-5-phosphate-3-epimerase, transketolase, and transaldolase. It is preferable that one or more genes encoding such enzymes be introduced. It is more preferable to introduce two or more such genes in combination, further preferable to introduce three or more genes in combination, and the most preferable to introduce all of the genes above.

More specifically, the xylulokinase (XK) gene of any origin can be used without particular limitation. A wide variety of microorganisms, such as bacteria and yeasts, which assimilate xylulose, possess the XK gene. Information concerning XK genes can be obtained by searching the website of NCBI or other institutions, according to need. Preferable examples of such genes include the XK genes derived from yeasts, lactic acid bacteria, E. coli bacteria, and plants. An example of an XK gene is XKS1, which is an XK gene derived from the S. cerevisiae S288C strain (GenBank: Z72979) (the nucleotide sequence and the amino acid sequence in the CDS coding region).

More specifically, a transaldolase (TAL) gene, a transketolase (TKL) gene, a ribulose-5-phosphate epimerase (RPE) gene, and a ribose-5-phosphate ketoisomerase (RKI) gene of any origin can be used without particular limitation. A wide variety of organisms comprising the pentose phosphate pathway possess such genes. For example, a common yeast such as S. cerevisiae possesses such genes. Information concerning such genes can be obtained from the website of NCBI or other institutions, according to need. Genes derived from the same genus as the host eukaryotic cells, such as eukaryotic or yeast cells, are preferable, and genes originating from the same species as the host eukaryotic cells are further preferable. A TAL1 gene, a TKL1 gene and a TKL2 gene, an RPE1 gene, and an RKI1 gene can be preferably used as the TAL gene, the TKL genes, the RPE gene, and the RKI gene, respectively. Examples of such genes include a TAL1 gene derived from the S. cerevisiae S288 strain (GenBank:U19102), a TKL1 gene derived from the S. cerevisiae S288 strain (GenBank: X73224), an RPE1 gene derived from the S. cerevisiae S288 strain (GenBank: X83571), and an RKI1 gene derived from the S. cerevisiae S288 strain (GenBank: Z75003).

[Production of Recombinant Yeast]

The acetaldehyde dehydrogenase gene as described above is introduced into a host yeast genome, an enzyme involved with trehalose accumulation is regulated in such yeast, and the recombinant yeast according to the present invention can be produced. The acetaldehyde dehydrogenase gene may be introduced into a yeast that does not have xylose-metabolizing ability, a yeast that inherently has xylose-metabolizing ability, or a yeast that does not have xylose-metabolizing ability together with the xylose metabolism-associated gene. When the acetaldehyde dehydrogenase gene and the genes described above are introduced into a yeast, such genes may be simultaneously introduced thereinto, or such genes may be successively introduced with the use of different expression vectors.

Examples of host yeasts that can be used include, but are not particularly limited to, Candida Shehatae, Pichia stipitis, Pachysolen tannophilus, Saccharomyces cerevisiae, and Schizosaccaromyces pombe, with Saccharomyces cerevisiae being particularly preferable. Experimental yeast strains may also be used from the viewpoint of experimental convenience, or industrial (practical) strains may also be used from the viewpoint of practical usefulness. Examples of industrial strains include yeast strains used for the production of wine, sake, and shochu.

Use of a host yeast having homothallic properties is preferable. According to the technique disclosed in JP 2009-34036 A, multiple copies of genes can be easily introduced into a genome with the use of a yeast having homothallic properties. The term “yeast having homothallic properties” has the same meaning as the term “homothallic yeast.” Yeasts having homothallic properties are not particularly limited, and any yeasts can be used. An example of a yeast having homothallic properties is the Saccharomyces cerevisiae OC-2 strain (NBRC2260), although yeasts are not limited thereto. Examples of other yeasts having homothallic properties include an alcohol-producing yeast (Taiken No. 396, NBRC0216) (reference: “Alcohol kobo no shotokusei” (“Various properties of alcohol-producing yeast”), Shuken Kaiho, No. 37, pp. 18-22, 1998.8), an ethanol-producing yeast isolated in Brazil and in Okinawa. Japan (reference: “Brazil to Okinawa de bunri shita Saccharomyces cerevisiae yaseikabu no idengakuteki seishitsu” (“Genetic properties of wild-type Saccharomyces cerevisiae isolated in Brazil and in Okinawa”), the Journal of the Japan Society for Bioscience, Biotechnology, and Agrochemistry, Vol. 65, No. 4, pp. 759-762, 1991.4), and 180 (reference: “Alcohol Hakkoryoku no tsuyoi kobo no screening” (“Screening of yeast having potent alcohol-fermenting ability”), the Journal of the Brewing Society of Japan, Vol. 82, No. 6, pp. 439-443, 1987.6). In addition, the HO gene may be introduced into a yeast exhibiting heterothallic phenotypes in an expressible manner, and the resulting yeast can be used as a yeast having homothallic properties. That is, the term “yeast having homothallic properties” used herein also refers to a yeast into which the HO gene has been introduced in an expressible manner.

The Saccharomyces cerevisiae OC-2 strain is particularly preferable since it has heretofore been used for wine brewing and the safety thereof has been verified. As described in the Examples below, the Saccharomyces cerevisiae OC-2 strain is preferable in terms of its excellent promoter activity at high sugar concentrations. In particular, the Saccharomyces cerevisiae OC-2 strain is preferable in terms of its excellent promoter activity for the pyruvate decarboxylase gene (PDC1) at high sugar concentrations.

Promoters of genes to be introduced are not particularly limited. For example, promoters of the glyceraldehyde 3 phosphate dehydrogenase gene (TDH3), the 3-phosphoglycerate kinase gene (PGK 1), and the high-osmotic pressure response 7 gene (HOR7) can be used. The promoter of the pyruvate decarboxylase gene (PDC1) is particularly preferable in terms of its high capacity for expressing target genes in a downstream region at high levels.

Specifically, the genes as described above may be introduced into the yeast genome together with an expression-regulating promoter or another expression-regulated region. Such gene may be introduced into a host yeast genome in such a manner that expression thereof is regulated by a promoter or another expression-regulated region of a gene that is inherently present therein.

The genes as described above can be introduced into the genome by any conventional technique known as a yeast transformation technique. Specific examples include, but are not limited to, electroporation (Meth. Enzym., 194, p. 182, 1990), the spheroplast technique (Proc. Natl. Acad. Sci., U.S.A., 75, p. 1929, 1978), and the lithium acetate method (J. Bacteriology, 153, p. 163, 1983; Proc. Natl. Acad. Sci., U.S.A., 75, p. 1929, 1978; and Methods in yeast genetics, 2000 Edition: A Cold Spring Harbor Laboratory Course Manual).

[Production of Ethanol]

The method for ethanol production according to the present invention is a method for synthesizing ethanol from a saccharide source contained in a medium with the use of the recombinant yeast described above. According to the method for ethanol production according to the present invention, the recombinant yeast can metabolize acetic acid contained in a medium, and acetic acid concentration in a medium is lowered in association with ethanol fermentation.

When producing ethanol with the use of the recombinant yeast described above, ethanol fermentation is carried out by culture in a medium containing at least either or both of glucose and xylose. A medium in which ethanol fermentation is carried out contains at least either or both of glucose and xylose as a carbon source (or carbon sources). The medium may contain another carbon source.

Either or both of glucose and xylose that are contained in a medium to be used for ethanol fermentation can be derived from a cellulosic biomass. The cellulosic biomass may have been subjected to a conventional pretreatment technique. Examples of pretreatment techniques include, but are not particularly limited to, degradation of a lignin with a microorganism and grinding of a cellulosic biomass. For example, a ground cellulosic biomass may be subjected to pretreatment, such as soaking thereof in a dilute sulfuric acid solution, alkaline solution, or ionic solution, hydrothermal treatment, or fine grinding. Thus, the efficiency of biomass saccharification can be improved.

In other words, the medium may comprise cellulose such as a cellulosic biomass and cellulase. In such a case, the medium contains glucose generated by cellulase reacting on cellulose.

The medium may further comprise a cellulosic biomass and hemicellulase that saccharifies hemicellulose contained in a cellulosic biomass to generate xylose. In such a case, the medium contains xylose generated when hemicellulase acts on hemicellulose.

A saccharified solution resulting from saccharification of a cellulosic biomass may be added to a medium used for ethanol fermentation. In such a case, the saccharified solution may contain cellulose or cellulase remained and glucose generated, or it may contain hemicellulose or hemicellulase remained and xylose generated.

As described above, the method for ethanol production according to the present invention comprises a process of ethanol fermentation carried out with the use of at least either or both glucose and xylose as a saccharide source (or saccharide sources). According to the method for ethanol production of the present invention, ethanol can be produced via ethanol fermentation carried out with the use of either or both glucose and xylose as a saccharide source (or saccharide sources). According to the method for ethanol production with the use of the recombinant yeast according to the present invention, ethanol fermentation is followed by recovery of ethanol from the medium. Ethanol may be recovered by any conventional means without particular limitation. After the completion of the process of ethanol fermentation mentioned above, for example, a liquid layer containing ethanol is separated from a solid layer containing the recombinant yeast or solid matter via solid-solution separation. Thereafter, ethanol contained in a liquid layer is separated and purified by distillation, so that highly purified ethanol can be recovered. The degree of ethanol purification can be adequately determined in accordance with the purpose of use of the ethanol.

When producing ethanol with the use of a saccharide derived from a biomass, in general, a fermentation inhibitor, such as acetic acid or furfural, may occasionally be generated in the process of pretreatment or saccharification. In particular, acetic acid is known to inhibit the growth and multiplication of yeasts and to lower the efficiency for ethanol fermentation conducted with the use of xylose as a saccharide source.

According to the present invention, however, recombinant yeasts resulting from introduction of the acetaldehyde dehydrogenase gene and regulation of an enzyme involved with trehalose accumulation, so as to increase the amount of trehalose accumulated in a cell, are used. Thus, acetic acid contained in a medium can be metabolized, and acetic acid concentration in a medium can be maintained at a low level. Accordingly, the method for ethanol production according to the present invention can achieve an ethanol yield superior to that achieved with the use of a yeast that has not experienced introduction of the acetaldehyde dehydrogenase gene or regulation of an enzyme involved with trehalose accumulation, so as to increase the amount of trehalose accumulated in a cell.

According to the method for ethanol production according to the present invention, acetic acid concentration in a medium remains low after the recombinant yeast has been cultured for a given period of time. Even if part of the medium after such given period of time is used for a continuous culture system in which a new culture process is initiated, accordingly, the amount of acetic acid carry-over can be reduced. According to the method for ethanol production according to the present invention, therefore, the amount of acetic acid carry-over can be reduced even when cells are recovered and reused after the completion of the process of ethanol fermentation.

The method for ethanol production according to the present invention may employ the so-called simultaneous saccharification and fermentation process, in which the step of saccharification of cellulose contained in a medium with a cellulase proceeds simultaneously with the process of ethanol fermentation carried out with the use of xylose and glucose generated upon saccharification as saccharide sources. With the simultaneous saccharification and fermentation process, the step of saccharification of a cellulosic biomass is carried out simultaneously with the process of ethanol fermentation.

Methods of saccharification are not particularly limited, and, for example, an enzymatic method involving the use of a cellulase preparation, such as cellulase or hemicellulase, may be employed. A cellulase preparation contains a plurality of enzymes involved in degradation of a cellulose chain and a hemicellulose chain, and it exhibits a plurality of types of activity, such as endoglucanase activity, endoxylanase activity, cellobiohydrolase activity, glucosidase activity, and xylosidase activity. Cellulase preparations are not particularly limited, and examples include cellulases produced by Trichoderma reesei and Acremonium cellulolyticus. Commercially available cellulase preparations may also be used.

In the simultaneous saccharification and fermentation process, a cellulase preparation and the recombinant yeast as described above are added to a medium containing a cellulosic biomass (a biomass after pretreatment may be used), and the recombinant yeast is cultured at a given temperature range. Culture may be carried out at any temperature without particular limitation, and the temperature may be 25 degrees C. to 45 degrees C., and preferably 30 degrees C. to 40 degrees C., from the viewpoint of ethanol fermentation efficiency. The pH level of the culture solution is preferably 4 to 6. When conducting culture, stirring or shaking may be carried out. Alternatively, the simultaneous saccharification and fermentation process may be carried out irregularly in such a manner that saccharification is first carried out at an optimal temperature for an enzyme (40 degrees C. to 70 degrees C.), temperature is lowered to a given level (30 degrees C. to 40 degrees C.), and a recombinant yeast is then added thereto.

### EXAMPLES

Hereafter, the present invention is described in greater detail with reference to the Examples, although the technical scope of the present invention is not limited to these Examples.

### Example 1

In the present Example, a recombinant yeast resulting from introduction of the acetaldehyde dehydrogenase gene and disruption of the NTH1 gene involved with trehalose accumulation was prepared. The acetic acid-metabolizing ability of the resulting recombinant yeast was evaluated.

[Preparation of Vector for Gene Introduction]

(1) Plasmid for XI, XKS1, TKL1, TAL1, RKI1, and RPE1 Gene Introduction and GRE3 Gene Disruption

A plasmid, pUC-5U_GRE3-P_HOR7-TKL1-TAL1-FBA1_P-P_ADH1-RPE1-RKI1-TEF_P-P_TDH1-XI_N337C-T_DIT1-P_TDH3-XKS1-T_HIS3-LoxP-G418-LoxP-3U_GRE3, was prepared. This plasmid was constructed to comprise, at the GRE3 gene locus, a sequence necessary for disruption of the GRE3 gene and introduction of the following genes into yeast: a mutated gene for which the rate of xylose assimilation has been improved as a result of substitution of asparagine at amino acid position 337 of the xylose isomerase gene derived from the intestinal protozoa of Reticulitermes speratus (nucleotide sequence: SEQ ID NO: 11; amino acid sequence: SEQ ID NO: 12) with cysteine (XI_N337C; SEQ ID NO: 48 in WO 2014/156194); a yeast-derived xylulokinase (XKS1) gene (nucleotide sequence: SEQ ID NO: 17; amino acid sequence: SEQ ID NO: 18); a transketolase 1 (TKL1) gene (nucleotide sequence: SEQ ID NO: 19; amino acid sequence: SEQ ID NO: 20), a transaldolase 1 (TAL1) gene (nucleotide sequence: SEQ ID NO: 21; amino acid sequence: SEQ ID NO: 22), a ribulose phosphate epimerase 1 (RPE1) gene (nucleotide sequence: SEQ ID NO: 23; amino acid sequence: SEQ ID NO: 24) and a ribose phosphate ketoisomerase (RKI1) gene (nucleotide sequence: SEQ ID NO: 25; amino acid sequence: SEQ ID NO: 26) of the pentose phosphate circuit.

This plasmid was constructed to comprise: the TKL1 gene derived from the Saccharomyces cerevisiae BY4742 strain in which an HOR7 promoter is added on the 5′ side; the TAL1 gene in which an FBA1 promoter is added; the RKI1 gene in which an ADH1 promoter is added; the RPE1 gene in which a TEF1 promoter is added; XI_N337C in which a TDH1 promoter and a DIT1 terminator are added (prepared through the total synthesis on the basis of a sequence designed by changing codons over the entire region in accordance with the frequency of codon usage of the yeast); the XKS1 gene in which a TDH3 promoter and an HIS3 terminator are added; a gene sequence (5U_GRE3) comprising an upstream region of approximately 700 bp from the 5′ terminus of the GRE3 gene and a DNA sequence (3U_GRE3) comprising a downstream region of approximately 800 bp from the 3′ terminus of the GRE3 gene, which are regions to be integrated into the yeast genome via homologous recombination; and a gene sequence (G418 marker) comprising the G418 gene, which is a marker. The LoxP sequence was introduced on the both sides of the marker gene, thereby making it possible to remove the marker.

Each DNA sequence can be amplified via PCR with the use of the primers shown in Table 1. In order to allow DNA fragments to bind to each other, a DNA sequence was added to a primer so as to overlap with an adjacent DNA sequence by about 15 bp, a target DNA fragment thereof was amplified with the use of the genomic DNA of the Saccharomyces cerevisiae BY4742 strain, DNA of the XI_N337C synthetic gene, and synthetic DNA of the LoxP sequence as templates, the DNA fragments were successively bound to each other with the use of In-Fusion (registered trademark) HD Cloning Kit (Takara Bio), and the resultant was then cloned into the pUC19 plasmid. Thus, the plasmid meeting the final objective was prepared.

(2) Plasmid for mhpF and ADH1 Gene Introduction and ADH2 Gene Disruption

A plasmid, pUC5U_ADH2-P_TDH3-ADH1-T_ADH1-DIT1_T-mhpF-HOR7_P-URA3-3U_ADH2, was prepared. This plasmid was constructed to comprise, at the ADH2 gene locus, a sequence necessary for disruption of the ADH2 gene (nucleotide sequence: SEQ ID NO: 15; and amino acid sequence: SEQ ID NO: 16) and introduction of the following genes into yeast: the acetaldehyde dehydrogenase gene (mhpF) derived from E. coli (nucleotide sequence: SEQ ID NO: 1; and amino acid sequence: SEQ ID NO: 2) and the yeast-derived alcohol dehydrogenase 1 (ADH1) gene (nucleotide sequence: SEQ ID NO: 13; and amino acid sequence: SEQ ID NO: 14).

This plasmid was constructed to comprise: the ADH1 gene derived from the Saccharomyces cerevisiae BY4742 strain in which a TDH3 promoter is added on the 5′ side; the mhpF gene in which an HOR7 promoter and a DIT1 terminator are added (NCBI Accession NO: 945008; prepared through the total synthesis on the basis of a sequence designed by changing codons over the entire region in accordance with the frequency of codon usage of the yeast); a gene sequence (5U_ADH2) comprising an upstream region of approximately 700 bp from the 5′ terminus of the ADH2 gene and a DNA sequence (3U_ADH2) comprising a downstream region of approximately 800 bp from the 3′ terminus of the ADH2 gene, which are regions to be integrated into the yeast genome via homologous recombination; and a gene sequence (URA3 marker) comprising the URA3 gene, which is a marker.

Each DNA sequence can be amplified via PCR with the use of the primers shown in Table 1. In order to allow DNA fragments to bind to each other, a DNA sequence was added to a primer so as to overlap with an adjacent DNA sequence by about 15 bp, a target DNA fragment thereof was amplified with the use of the genomic DNA of the Saccharomyces cerevisiae BY4742 strain or DNA of the mhpF synthetic gene as a template, the DNA fragments were successively bound to each other with the use of In-Fusion (registered trademark) HD Cloning Kit, and the resultant was then cloned into the pUC19 plasmid. Thus, the plasmid meeting the final objective was prepared.

(3) Plasmid for adhE and ADH1 Gene Introduction and ADH2 Gene Disruption

A plasmid, pUC5U_ADH2-P_TDH3-ADH1-T_ADH1-DIT1_T-adhE-HOR7_P-URA3-3U_ADH2, was prepared. This plasmid was constructed to comprise, at the ADH2 gene locus, a sequence necessary for disruption of the ADH2 gene (nucleotide sequence: SEQ ID NO: 15; amino acid sequence: SEQ ID NO: 16) and introduction of the following genes into yeast: the acetaldehyde dehydrogenase gene (adhE) derived from E. coli (nucleotide sequence: SEQ ID NO: 3; amino acid sequence: SEQ ID NO: 4) and the yeast-derived alcohol dehydrogenase 1 (ADH1) gene (nucleotide sequence: SEQ ID NO: 13; amino acid sequence: SEQ ID NO: 14).

This plasmid was constructed to comprise: the ADH1 gene derived from the Saccharomyces cerevisiae BY4742 strain in which a TDH3 promoter is added on the 5′ side; the adhE gene in which an HOR7 promoter and a DIT1 terminator are added (NCBI Accession NO: 945837; prepared through the total synthesis on the basis of a sequence designed by changing codons over the entire region in accordance with the frequency of codon usage of the yeast); a gene sequence (5U_ADH2) comprising an upstream region of approximately 700 bp from the 5′ terminus of the ADH2 gene and a DNA sequence (3U_ADH2) comprising a downstream region of approximately 800 bp from the 3′ terminus of the ADH2 gene, which are regions to be integrated into the yeast genome via homologous recombination; and a gene sequence (URA3 marker) comprising the URA3 gene, which is a marker.

Each DNA sequence can be amplified via PCR with the use of the primers shown in Table 1. In order to allow DNA fragments to bind to each other, a DNA sequence was added to a primer so as to overlap with an adjacent DNA sequence by about 15 bp, a target DNA fragment thereof was amplified with the use of DNA of the pUC5U_ADH2-P_TDH3-ADH1-T_ADH1-DIT1_T-mhpF-HOR7_P-URA3-3U_ADH2 plasmid or the adhE synthetic gene as a template, the DNA fragments were successively bound to each other with the use of In-Fusion (registered trademark) HD Cloning Kit, and the resultant was then cloned into the pUC19 plasmid. Thus, the plasmid meeting the final objective was prepared.

(4) Plasmid for eutE and ADH1 Gene Introduction and ADH2 Gene Disruption

A plasmid, pUC5U_ADH2-P_TDH3-ADH1-T_ADH-DIT1_T-eutE-HOR7_P-URA3-3U_ADH2, was prepared. This plasmid was constructed to comprise, at the ADH2 gene locus, a sequence necessary for disruption of the ADH2 gene (nucleotide sequence: SEQ ID NO: 15; amino acid sequence: SEQ ID NO: 16) and introduction of the following genes into yeast: the acetaldehyde dehydrogenase gene (eutE) derived from E. coli (nucleotide sequence: SEQ ID NO: 5: amino acid sequence: SEQ ID NO: 6) and the yeast-derived alcohol dehydrogenase 1 (ADH1) gene (nucleotide sequence: SEQ ID NO: 13; amino acid sequence: SEQ ID NO: 14).

This plasmid was constructed to comprise: the ADH1 gene derived from the Saccharomyces cerevisiae BY4742 strain in which a TDH3 promoter is added on the 5′ side; the eutE gene in which the HOR7 promoter and the DIT1 terminator are added (NCBI Accession NO: 946943, prepared through the total synthesis on the basis of a sequence designed by changing codons over the entire region in accordance with the frequency of codon usage of the yeast); a gene sequence (5U_ADH2) comprising an upstream region of approximately 700 bp from the 5′ terminus of the ADH2 gene and a DNA sequence (3U_ADH2) comprising a downstream region of approximately 800 bp from the 3′ terminus of the ADH2 gene, which are regions to be integrated into the yeast genome via homologous recombination; and a gene sequence (URA3 marker) comprising the URA3 gene, which is a marker.

Each DNA sequence can be amplified via PCR with the use of the primers shown in Table 1. In order to allow DNA fragments to bind to each other, a DNA sequence was added to a primer so as to overlap with an adjacent DNA sequence by about 15 bp, a target DNA fragment thereof was amplified with the use of the plasmid pUC5U_ADH2-P_TDH3-ADH1-T_ADH-DIT1_T-mhpF-HOR7_P-URA3-3U_ADH2 or DNA of the eutE synthetic gene as a template, the DNA fragments were successively bound to each other with the use of In-Fusion (registered trademark) HD Cloning Kit, and the resultant was then cloned into the pUC19 plasmid. Thus, the plasmid meeting the final objective was prepared.

(5) Plasmid for NTH1 Gene Disruption

A plasmid, pCR-5U_NTH1U-LoxP-G418-LoxP-3U_NTH1, comprising a sequence necessary for disruption of the NTH1 gene was prepared.

This plasmid was constructed to comprise: a DNA sequence (5U_NTH1) comprising an upstream region of approximately 1050 bp of the NTH1 gene and a DNA sequence (3U_NTH1) comprising a downstream region of approximately 1050 bp of the NTH1 gene, which are regions to be integrated into the yeast genome via homologous recombination and to disrupt the neutral trehalase gene (NTH1): and a gene sequence (G418 marker) comprising the G418 gene, which is a marker.

Each DNA sequence can be amplified via PCR with the use of the primers shown in Table 1. In order to allow DNA fragments to bind to each other, a DNA sequence was added to a primer so as to overlap with an adjacent DNA sequence by about 15 bp, a target DNA fragment was amplified with the use of the plasmid (pUC-5U_GRE3-P_HOR7-TKL1-TAL1-FBA1_P-P_ADH1-RPE1-RKI-TEF1_P-P_TDH1-XI_N337C-T_DIT1-P_TDH3-XKS1-T_HIS3-LoxP-G418-LoxP-3U_GRE3) or genomic DNA of the yeast OC2 strain as a template, the DNA fragments were successively bound to each other with the use of In-Fusion (registered trademark) HD Cloning Kit, and the resultant was then cloned into the pUC19 plasmid. Thus, the plasmid meeting the final objective was prepared.

(6) Plasmid for Substitution of NTH1 Gene Terminator

A plasmid, pUC5U_NTH1-NTH1-T_GIC1-LoxP-P_CYC1-G418-T_URA3-LoxP-3U_NTH1, comprising a sequence necessary for substitution of the NTH1 gene terminator with GIC1, which is a terminator whose expression level is low (US20130244243), was prepared.

This plasmid was constructed to comprise: a DNA sequence (5U_NTH1) comprising an upstream region of approximately 1050 bp of the NTH1 gene. ORF of NTH1, and a DNA sequence (3U_NTH1) comprising a downstream region of approximately 1050 bp of the NTH1 gene, which are regions to be integrated into the yeast genome via homologous recombination and to substitute the neutral trehalase gene (NTH1) terminator; and a gene sequence (G418 marker) comprising the G418 gene, which is a marker.

Each DNA sequence can be amplified via PCR with the use of the primers shown in Table 1. In order to allow DNA fragments to bind to each other, a DNA sequence was added to a primer so as to overlap with an adjacent DNA sequence by about 15 bp, a target DNA fragment thereof was amplified with the use of the plasmid (pUC-5U_GRE3-P_HOR7-TKL1-TAL1-FBA1_P-P_ADH1-RPE1-RKI1-TEF1_P-P_TDH1-XI_N337C-T_DIT1-P_TDH3-XKS1-T_HIS3-LoxP-G418-LoxP-3U_GRE3) or genomic DNA of the yeast OC2 strain as a template, the DNA fragments were successively bound to each other with the use of In-Fusion (registered trademark) HD Cloning Kit, and the resultant was then cloned into the pUC19 plasmid. Thus, the plasmid meeting the final objective was prepared.

(7) Fragment for URA3 Gene Introduction

A fragment of the wild-type URA3 gene used for transforming the non-functioning URA3 gene at the URA3 gene locus into a wild-type gene via homologous recombination was amplified from the OC2 strain. This DNA fragment can be amplified via PCR using primers shown in Table 1.

(8) Plasmid for Cre Gene Expression

A plasmid, pYES-Cre, used to express multiple copies of the Cre genes was prepared.

This plasmid was constructed by introducing the Cre gene (NCBI Accession NO: NP_415757.1, prepared through the total synthesis on the basis of a sequence designed by changing codons over the entire region in accordance with the frequency of codon usage of the yeast) fused to the GAL1 promoter, which is induced with galactose, into pYES6/CT (Life Technologies Corporation).

Each DNA sequence necessary for construction can be amplified with the use of the primers shown in Table 1. In order to allow DNA fragments to bind to each other, a DNA sequence was added to the primer shown in Table 1 so as to overlap with an adjacent DNA sequence by about 15 bp, a target DNA fragment thereof was amplified with the use of the plasmid (YES6/CT) or DNA of the Cre synthesizing gene as a template, the DNA fragments were bound to each other with the use of InFusion (registered trademark) HD Cloning Kit. Thus, the plasmid meeting the final objective was prepared.

TABLE 1
Primers used for each amplified fragment

| | | SEQ ID |
|---|---|---|
| Amplified DNA fragment | Primer sequence (5′-3′) | NO: |
| pUC-5U_GRE3-P_HOR7-TKL1-TAL1-FBA1_P-P_ADH1-RPE1-RKI1-TEF1-P-P_TDH1-XI_N337C-T_DIT1- | | |
| P_TDH3-XKS1-T_HIS3-LoxP-G418-LoxP-3U_GRE3 | | |
| 5U_GRE3 | 5′-TGGGAATATTACCGCTCGAAG-3′ | 27 |
| | 5′-CTTTAAAAAATTTCCAATTTTCCTTTACG-3′ | 28 |
| HOR7 promoter | 5′-GGAAATTTTTTAAAGTCGCAGCCACGGGTCAAC-3′ | 29 |
| | 5′-GTGAATTGAGTCATTTTTTATTATTAGTCTTTTTTTTTTTTGACAA | 30 |
| | TATC-3′ | |
| TKL1 | 5′-ATGACTCAATTCACTGACATTGATAAGCTAG-3′ | 31 |
| (including a terminator region) | 5′-CCTTAAATCAACGTCATATTCTTTATTGGCTTTATAC-3′ | 32 |
| TAL1 | 5′-GACGTTGATTTAAGGTGGTTCCGG-3′ | 33 |
| (including a terminator region) | 5′-ATGTCTGAACCAGCTCAAAAGAAAC-3′ | 34 |
| FBA1 promoter | 5′-AGCTGGTTCAGACATTTTGAATATGTATTACTTGGTTATGGTTAT | 35 |
| | ATATGAC-3′ | |
| | 5′-ACTGGTAGAGAGCGACTTTGTATGC-3′ | 36 |
| ADH1 promoter | 5′-CAAAGTCGCTCTCTACCAGTCGCTTTCAATTCATTTGGGTG-3′ | 37 |
| | 5′-TGTATATGAGATAGTTGATTGTATGC-3′ | 38 |
| RPE1 | 5′-ACTATCTCATATACAATGGTCAAACCAATTATAGCTCCC-3′ | 39 |
| (including a terminator region) | 5′-AAATGGATATTGATCTAGATGGCGG-3′ | 40 |
| RKI1 | 5-GATCAATATCCATTTCTTGGTGTGTCATCGGTAGTAACGCC-3′ | 41 |
| (including a terminator region) | 5′-AGTTTTAATTACAAAATGGCTGCCGGTGTCCCAAA-3′ | 42 |
| TEF1 promoter | 5′-TTGTAATTAAAACTTAGATTAGATTGCTATGCTTTC-3′ | 43 |
| | 5′-AGGAACAGCCGTCAAGGG-3′ | 44 |
| TDH1 promoter | 5′-TTGACGGCTGTTCCTCTTCCCTTTTACAGTGCTTC-3′ | 45 |
| | 5′-TTTGTTTTGTGTGTAAATTTAGTGAAGTACTG-3′ | 46 |
| Xi_N337C | 5′-TACACACAAAACAAAATGTCTCAAATTTTTAAGGATATCCC-3′ | 47 |
| | 5′-AGCGCTCTTACTTTAGCGATCGCACTAGTTTATTGAAAC-3′ | 48 |
| DIT1 terminator | 5′-TAAAGTAAGAGCGCTACATTGGTCTACC-3′ | 49 |
| | 5′-TAACATTCAACGCTATTACTCCGCAACGCTTTTCTG-3′ | 50 |
| TDH3 promoter | 5′-TAGCGTTGAATGTTAGCGTCAACAAC-3′ | Si |
| | 5′-TTTGTTTGTTTATGTGTGTTTATTCGAAACTAAGTTCTTGG-3′ | 52 |
| XKS1 | 5′-ACATAAACAAACAAAATGTTGTGTTCAGTAATTCAGAGACAG-3′ | 53 |
| | 5′-AAATAATCGGTGTCATTAGATGAGAGTCTTTTCCAGTTC-3′ | 54 |
| HIS3 terminator | 5′-TGACACCGATTATTTAAAGCTGCAG-3′ | 55 |
| | 5′-ACAGCGCGCCTCGTTCAG-3′ | 55 |
| LoxP | 5′-AACGAGGCGCGCTCTAATTCCGCTGTATAGCTC-3′ | 57 |
| (including a linker sequence) | 5′-ATAATGTATGCTATACGAAGTTATAGGGAAAGATATGAGCTATAC- | 58 |
| | 3′ | |
| CYC1 promoter | 5′-TATAGCATACATTATACGAAGTTATACGACATCGTCGAATATG-3′ | 59 |
| | 5′-TATTAATTTAGTGTGTGTATTTGTGTTTGTGTG-3′ | 60 |
| G184 | 5′-CACACTAAATTAATAATGAGCCATATTCAACGGG-3′ | 61 |
| | 5′-TTTAGTAGACATGCATTACAACCAATTAACCAATTCTG-3′ | 62 |
| URA3 terminator | 5′-TGCATGTCTACTAAACTCACAAATTAGAGCTTCAATT-3′ | 63 |
| | 5′-ATAATGTATGCTATACGAAGTTATGGGTAATAACTGATATAATTAA | 64 |
| | ATTGAAGC-3′ | |
| LoxP | 5′-TATAGCATACATTATACGAAGTTATIGACACCGATTATTTAAAGC | 65 |
| (including a linker sequence) | TG-3′ | |
| | 5′-ATTTTACTGGCTGGACTATGCTGCAGCTTTAAATAATCG-3′ | 66 |
| 3U_GRE3 | 5′-TCCAGCCAGTAAAATCCATACTCAAC-3′ | 67 |
| | 5′-GTCTTTTTGCCAGCCAGTCC-3′ | 68 |
| pUC19 | 5′-CACACCTTCCCCCTTGATCCTCTAGAGTCGACC-3′ | 69 |
| | 5′-GCGGTAATTCCCAGATCCCCGGGTACCGAGCTC-3′ | 70 |
| pUC-5U_ADH2-P_TDH3-ADH1-T_ADH1-DIT1_T-mhpF-HOR7_P-URA3-3U_ADH2 | | |
| 5U_ADH2 | 5′-CGGTACCCGGGGATCCTATGGGACTTCCGGGAA-3′ | 71 |
| | 5′TAACATTCAACGCTATGTGTATTACGATATAGTTAATAGTTGATA | 72 |
| | G-3′ | |
| TDH3 promoter | 5′-TAGCGTTGAATGTTAGCGTCAACAAC-3′ | 73 |
| | 5′-TTTGTTTGTTTATGTGTGTTTATTCGAAACTAAGTTCTTGG-3′ | 74 |
| ADH1 | 5′-ACATAAACAAACAAAATGTCTATCCCAGAAACTCAAAAAG-3′ | 75 |
| (inciuding a terminator region) | 5′-TTGTCCTCTGAGGACATAAAATACACACCG-3′ | 76 |
| DIT1 terminator | 5′-GTCCTCAGAGGACAATTACTCCGCAACGCTTTTC-3′ | 77 |
| | 5′-GGAGAGGCCGCATAATAAAGTAAGAGCGCTACATTGG-3′ | 78 |
| mhpF | 5′-TTATGCGGCCTCTCCTGC-3′ | 79 |
| | 5′-AGACTAATAATAAAAATGTCAAAGAGAAAAGTTGCTATTATCG-3′ | 80 |
| HOR7 promoter | 5′-TTTTTATTATTAGTCTTTTTTTTTTTTGACAATATCTGTATGATTT | 81 |
| | G-3′ | |
| | 5′-GGAGATTACCGAATCTCGCTCGCAGCCACGGGT-3′ | 82 |
| URA3 | 5′-GATTCGGTAATCTCCGAGCAG-3′ | 83 |
| (including promoter and terminator | 5′-ACATAAGAGATCCGCGGGTAATAACTGATATAATTAAATTG-3′ | 84 |
| regions) | | |
| 3U_ADH2 | 5′-GCGGATCTCTTATGTCTTTACGATTTATAGTTTTC-3′ | 85 |
| | 5′-GAGGGTTGGGCATTCATCAG-3′ | 86 |
| pUC19 | 5′-AATGCCCAACCCTCGATCCTCTAGAGTCGACC-3′ | 87 |
| | 5′-GATCCCCGGGTACCGAGC-3′ | 88 |
| pUC-5U_ADH2-P_TDH3-ADH1-T_ADH1-DIT1_T-adhE-HOR7_P-URA3-3U_ADH2 | | |
| Sequence other than adhE | 5′-TTTTATTATTAGTCTTTTTTTTTTTTGACAATATCTG-3′ | 89 |
| | 5′-TAAAGTAAGAGCGCTACATTGGTCTACC-3′ | 90 |
| adhE | 5′-AGCGCTCTTACTTTATTAAGCTGATTTCTTTGCTTTCTTC-3′ | 91 |
| | 5′-AGACTAATAATAAAAATGGCAGTTACGAACGTTGCAG-3′ | 92 |
| pUC-5U_ADH2-P_TDH3-ADH1-T_ADH1-DIT1_T-eutE-HOR7_P-URA3-3U_ADH2 | | |
| Sequence other than eutE | 5′-TTTTATTATTAGTCTTTTTTTTTTTTGACAATATCTG-3′ | 93 |
| | 5′-TAAAGTAAGAGCGCTACATTGGTCTACC-3′ | 94 |
| eutE | 5′-AGCGCTCTTACTTTACTAAACAATTCTGAATGCATCGAC-3′ | 95 |
| | 5′-AGACTAATAATAAAAATGAACCAACAAGACATAGAACAAG-3′ | 96 |
| pUC-5U_NTH1U-LoxP-G418-LoxP-3U_NTH1 | | |
| 5U_NTH1 | 5′-CAATATCTGCTGTACAAGCATACACC-3′ | 97 |
| | 5′-CTATACAGCGGAATTTTATGGTTATTTAACTGTAACGAATAGGCT | 98 |
| | AGC-3′ | |
| G418 marker | 5′-AATTCCGCTGTATAGCTCATATCTTTC-3′ | 99 |
| | 5′-GTATGCTGCAGCTTTAAATAATCGG-3′ | 100 |
| 3U_NTH1 | 5′-AAAGCTGCAGCATACCCTTATATCTATGCAGTTGGTTGTGAAAT | 101 |
| | C-3′ | |
| | 5′-GAAGGAACAGCTGGGCC-3′ | 102 |
| pUC19 | 5′-CCCAGCTGTTCCTTCGATCCTCTAGAGTCGACC-3′ | 103 |
| | 5′-GTACAGCAGATATTGGATCCCCGGGTACCGAGC-3′ | 104 |
| pUC-5U_NTH1-NTH1-T_GIC1-LoxP-P_CYC1-G418-T_URA3-LoxP-3U_NTH1 | | |
| 5U_NTH1 and NTH1 | 5′-CAATATCTGCTGTACAAGCATACACC-3′ | 105 |
| | 5′-AAGAAGAAAACTAGTCTATAGTCCATAGAGGTTTCTTTCTTG-3′ | 106 |
| GIC1 terminator | 5′-ACTAGTTTTCTTCTTTCCTCCTCTTCTTTG-3′ | 107 |
| | 5′-CGTTGGTTGAAACGTTGTCTG-3′ | 108 |
| G418 marker | 5′-ACGTTTCAACCAACGAATTCCGCTGTATAGCTCATATC-3′ | 109 |
| | 5′-GTATGCTGCAGCTTTAAATAATCGG-3′ | 110 |
| 3U_NTH1 | 5′-AAAGCTGCAGCATACCCTTATATCTATGCAGTTGGTTGTGAAAT | 111 |
| | C-3′ | |
| | 5′-GAAGGAACAGCTGGGCC-3 | 112 |
| pUC19 | 5′-CCCAGCTGTTCCTTCGATCCTCTAGAGTCGACC-3′ | 113 |
| | 5′-GTACAGCAGATATTGGATCCCCGGGTACCGAGC-3′ | 114 |
| Fragment for URA3 gene introduction | | |
| | 5′-AGGCTACTGCGCCAATTGAT-3′ | 115 |
| | 5′-TGCCCTACACGTTCGCTATG-3′ | 116 |
| pYES-Cre | | |
| pYES6/CT | 5′-GGTTTTTTCTCCTTGACGTTAAAGTATAG-3′ | 117 |
| | 5′-TTAGTTATGTCACGCTTACATTCACG-3′ | 118 |
| Cre | 5′-ATGTCTAACTTGTTGACTGTTC-3′ | 119 |
| | 5′-TCAATCACCATCTTCCAACAATC-3′ | 120 |

[Production of Yeast Strains Comprising Vectors Introduced Thereinto]

The diploid yeast, which is the Saccharomyces cerevisiae OC2 strain (NBRC2260), was selected in a 5-fluoroorotic acid-supplemented medium (Boeke, J. D., et al., 1987, Methods Enzymol., 154: 164-75.), and an uracil auxotrophic strain (OC2U) was designated as a host strain.

The yeast was transformed using the Frozen-EZ Yeast Transformation II (ZYMO RESEARCH) in accordance with the protocols included therein.

Regions between the homologous recombination sites of pUC5U_GRE3-P_HOR7-TKL-TAL1-FBA1_P-P_ADH1-RPE1-RKI1-TEF1_P-P_TDH1-XI_N337C-T_DIT1-P_TDH3-XKS1-T_HIS3-LoxP-G418-LoxP-3U_GRE3 were amplified by PCR, the resulting amplified fragments were used to transform the OC2U strain, the resulting transformants were applied to a G418-containing YPD agar medium, and the grown colonies were then subjected to acclimatization. The acclimatized elite strain was designated as the Uz1252 strain. This strain was applied to a sporulation medium (1% potassium phosphate, 0.1% yeast extract, 0.05% glucose, and 2% agar) for sporulation, and a diploid of the strain was formed by utilizing homothallism. The strain in which the mutated XI, TKL1, TAL1, RPE1, RKI1, and XKS1 genes had been incorporated into the GRE3 gene locus region of a diploid chromosome, and thus resulting in the disruption of the GRE3 gene, was obtained. The resulting strain was designated as the Uz1252 strain.

The plasmid for Cre gene expression was introduced into the Uz1252 strain, the G418 marker gene flanked by the LoxP sequences was removed via Cre/LoxP site-directed recombination, and the strain from which the Cre plasmid had been removed in the end was selected and designated as the Uz1252m strain.

Regions between homologous recombination sites of the plasmids: pUC5U_ADH2-P_TDH3-ADH1-T_ADH1-DIT1_T-mhpF-HOR7_P-URA3-3U_ADH2; pUC-5U_ADH2-P_TDH3-ADH1-T_ADH1-DIT1_T-adhE-HOR7_P-URA3-3U_ADH2; pUC-5U_ADH2-P_TDH3-ADH1-T_ADH-DIT1_T-eutE-HOR7_P-URA3-3U_ADH2, and pUC5U_NTH1-NTH1-T_GIC1-LoxP-P_CYC1-G418-T_URA3-LoxP-3U_NTH1, were amplified by PCR, the resulting amplified fragments and a fragment for URA3 gene introduction directly amplified from the genome of the OC2 strain were used to transform the Uz1252m strain, the resulting transformants were applied to a uracil-free SD agar medium or a G418-containing YPD agar medium, and the grown colonies were then subjected to acclimatization. The acclimatized elite strains were designated as the Uz1317 strain, the Uz1298 strain, the Uz1761 strain, the Uz1302 strain, and the Uz1313 strain.

Heterozygous recombination (1 copy) was observed in all of the above strains.

Sporulation was induced in a sporulation medium for the obtained Uz1317 strain, the Uz1298 strain, the Uz1302 strain, and the Uz1313 strain. Diploids of the strains formed by utilizing homothallism were designated as the Uz1319 strain, the Uz1318 strain, the Uz1346 strain, and the Uz1323 strain.

Regions between homologous recombination sites of the plasmid pCR-5U_NTH1U-LoxP-G418-LoxP-3U_NTH1 were amplified by PCR, the resulting fragments were used to transform the Uz1318 strain, the resulting transformants were applied to a G418-containing YPD agar medium, and the grown colonies were then subjected to acclimatization. The acclimatized elite strain was designated as the Uz1811 strain. The Uz1811 strain was applied to a sporulation medium for sporulation, and a diploid of the strain was formed by utilizing homothallism. The resulting strain was designated as the Uz1811dS strain.

Regions between homologous recombination sites of the plasmid pCR-5U_NTH1U-LoxP-G418-LoxP-3U_NTH1 were amplified by PCR, the resulting amplified fragments were used to transform the Uz1317 strain, the resulting transformants were applied to a G418-containing YPD agar medium, and the grown colonies were then subjected to acclimatization. The acclimatized elite strain was designated as the Uz1607 strain. The Uz1607 strain was applied to a sporulation medium for sporulation, and a diploid of the strain was formed by utilizing homothallism. Thus, the strain comprising diploids of 5U_ADH2-P_TDH3-ADH1-T_ADH1DIT1_T-mhpF-HOR7_P-URA3-3U_ADH2 and 5U_NTH1U-LoxP-G418-LoxP-3U_NTH1 introduced into both the ADH2 and NTH1 gene loci was obtained and designated as the Uz1607dS strain.

Regions between homologous recombination sites of the plasmid pCR-5U_NTH1U-LoxP-G418-LoxP-3U_NTH1 was amplified by PCR, the resulting fragments were used to transform the Uz1761 strain, the resulting transformants were applied to a G418-containing YPD agar medium, and the grown colonies were then subjected to acclimatization. The acclimatized elite strain was designated as the Uz1822 strain. The Uz1822 strain was applied to a sporulation medium for sporulation, and a diploid of the strain was formed by utilizing homothallism. As a result, a strain comprising diploids of 5U_ADH2-P_TDH3-ADH1-T_ADH1-DIT1_T-eutE-HOR7_P-URA3-3U_ADH2 and 5U_NTH1U-LoxP-G418-LoxP-3U_NTH11 introduced into both the ADH2 and NTH1 gene loci and a strain comprising a diploid of 5U_ADH2-P_TDH3-ADH1-T_ADH1-DIT1_T-eutE-HOR7_P-URA3-3U_ADH2 and the NTH1 gene locus transformed into its wild-type were obtained and designated as the Uz1822dS strain and the Uz1761dS strain.

Regions between homologous recombination sites of the plasmid pCR-5U_NTH1U-LoxP-G418-LoxP-3U_NTH1 or pUC5U_NTH1-NTH1-T_GIC1-LoxP-P_CYC1-G418-T_URA3-LoxP-3U_NTH1 were amplified by PCR, the resulting fragments were used to transform the Uz1323 strain, the resulting transformants were applied to a G418-containing YPD agar medium, and the grown colonies were then subjected to acclimatization. The acclimatized elite strains were designated as the Uz1662 strain and the Uz1661 strain. The Uz1662 strain and the Uz1661 strain were applied to a sporulation medium for sporulation, and diploids thereof were formed by utilizing homothallism. The resulting strains were designated as the Uz1662dS strain and the Uz1661dS strain.

Regions between the homologous recombination sites of the plasmid pUC5U_ADH2-P_TDH3-ADH1-T_ADH1-DIT1_T-mhpF-HOR7_P-URA3-3U_ADH2 were amplified by PCR, the resulting amplified fragments were used to transform the Uz1346 strain, the resulting transformants were applied to a G418-containing YPD agar medium, and the grown colonies were then subjected to acclimatization. The acclimatized elite strain was designated as the Uz1382 strain. The Uz1382 strain was applied to a sporulation medium for sporulation, a diploid of the strain was formed by utilizing homothallism, and the resulting strain was designated as the Uz1382dS strain. Genotypes of the strains produced as the final products are summarized in Table 2.

TABLE 2

| Strain | Genotype |
|---|---|
| Uz1252m | gre3:: XI_N337C XKS1 TKL1 TAL1 RKI1 RPE1 |
| Uz1319 | adh2:: mhpF ADH1 gre3:: XI_N337C XKS1 TKL1 TAL1 |
| | RKI1 RPE1 |
| Uz1318 | adh2:: adhE ADH1 gre3:: XI_N337C XKS1 TKL1 TAL1 |
| | RKI1 RPE1 |
| Uz1761dS | adh2:: eutE ADH1 gre3:: XI_N337C XKS1 TKL1 TAL1 |
| | RKI1 RPE1 |
| Uz1662dS | nth1:: G418 gre3:: XI_N337C XKS1 TKL1 TAL1 RKI1 |
| | RPE1 |
| Uz1607dS | adh2:: mhpF ADH1 nth1:: G418 gre3:: XI_N337C XKS1 |
| | TKL1 TAL1 RKI1 RPE1 |
| Uz1811dS | adh2:: adhE ADH1 nth1:: G418 gre3:: XI_N337C XKS1 |
| | TKL1 TAL1 RKI1 RPE1 |
| Uz1822dS | adh2:: eutE ADH1 nth1:: G418 gre3:: XI_N337C XKS1 |
| | TKL1 TAL1 RKI1 RPE1 |
| Uz1661dS | NTH1:: NTH1-GIC1t G418 gre3:: XI_N337C XKS1 TKL1 |
| | TAL1 RKI1 RPE1 |
| Uz1382dS | adh2:: mhpF ADH1 NTH1:: G418 gre3:: XI_N337C XKS1 |
| | TKL1 TAL1 RKI1 RPE1 |
| Uz1323 | gre3:: XI_N337C XKS1 TKL1 TAL1 RKI1 RPE1 |

[Fermentation Test]

From among the strains obtained in the manner described above, two strains exhibiting high fermentation ability were selected and subjected to a fermentation test in flasks in the manner described below.

At the outset, the test strains were introduced into 100-ml baffled flasks each containing 20 ml of YPD liquid medium containing glucose at 20 g/l (10 g/l yeast extract, 20 g/l peptone, and 20 g/l glucose), and culture was conducted at 30 degrees C. and 120 rpm for 24 hours. The strains were collected and introduced into 10-ml flasks each containing 8 ml of a medium for ethanol production (each medium has a different composition) (cell density: 0.3 g of dry cells/I). The fermentation test was carried out via shake culture (80 rpm; shake width: 35 mm; 30 degrees C.), or at temperature: 31 degrees C. or 34 degrees C. Each flask was stoppered with a rubber cap comprising a needle (inner diameter: 1.5 mm), and anaerobic conditions inside the flask were maintained by mounting a check valve at the tip of the needle.

Glucose, xylose, and ethanol in the fermentation liquor were assayed via HPLC (LC-10A; Shimadzu Corporation) under the conditions described below.

Column: Aminex HPX-87H

Mobile phase: 0.01N H₂SO₄

Flow rate: 0.6 ml/min

Temperature: 30 degrees C.

Detection apparatus: Differential refractometer (RID-10A)

[Results of Fermentation Test]

As shown in Tables 3 and 4, the control strain and the NTH1-disrupted strains did not assimilate acetic acid. In contrast, the strains in which ADH2 had been disrupted and ADH1 and acetaldehyde dehydrogenase had been overexpressed and the strains involving NTH1 disruption, in addition to ADH2 disruption and ADH1 and acetaldehyde dehydrogenase overexpression (i.e., Uz1607dS, Uz1811dS, and Uz1822dS strains), exhibited improvement in acetic acid assimilation.

TABLE 3

| | | | | Uz1607dS | | Uz1811dS | | Uz1822dS |
|---|---|---|---|---|---|---|---|---|
| | | | Uz1319 | adh2:: | Uz1318 | adh2:: | Uz1761dS | adh2:: |
| | | | adh2:: | mhpF | adh2:: | adhE | adh2:: | eutE |
| | Uz1323 | Uz1662dS | mhpF | ADH1 | adhE | ADH1 | eutE | ADH1 |
| | control | nth1 | ADH1 | nth1 | ADH1 | nth1 | ADH1 | nth1 |
| Acetic acid | 2.74 | 2.76 | 2.56 | 2.50 | 2.16 | 1.87 | 1.68 | 1.56 |
| concentration (g/L) | | | | | | | | |
| Acetic acid | −1.12 | −1.43 | 1.70 | 2.59 | 7.70 | 12.08 | 14.94 | 16.85 |
| assimilation rate (mg/h) | | | | | | | | |
| Ethanol | 40.33 | 41.56 | 40.00 | 42.33 | 46.14 | 49.76 | 45.88 | 49.59 |
| concentration (g/L) | | | | | | | | |

Medium: 50 g/l glucose, 100 g/l xylose, 10 g/l yeast extract, 20 g/l peptone, 2.7 g/l acetic acid, and 0.3 g/l furual
Fermentation duration: 46 hours
Fermentation temperature: 31° C.
Ethanol concentration, acetic acid concentration, and acetic acid assimilation rate: average values for 2 to 5 recombinant strains independently obtained

TABLE 4

| | | | | Uz1811dS |
|---|---|---|---|---|
| | | | Uz1318 | adh2:: |
| | | | adh2:: | adhE |
| | Uz1323 | Uz1662dS | adhE | ADH1 |
| | control | nth1 | ADH1 | nth1 |
| Acetic acid concentration | 3.54 | 3.52 | 3.27 | 2.78 |
| (g/L) | | | | |
| Acetic acid concentration | 0.024 | 0.027 | 0.15 | 0.052 |
| standard error | | | | |
| Acetic acid metabolizing rate | −0.655 | −0.339 | 3.457 | 10.97 |
| (mg/h) | | | | |
| Ethanol concentration | 27.5 | 24.7 | 30.2 | 40.4 |
| (g/L) | | | | |

Medium: 20 g/l glucose, 100 g/l xylose, 10 g/l yeast extract, 20 g/l peptone, 3.5 g/l acetic acid, and 0.3 g/l furfural
Fermentation duration: 46 hours
Fermentation temperature: 34° C.
Ethanol concentration, acetic add concentration, and acetic acid assimilation rate: average values for 5 recombinant strains independently obtained

All publications, patents, and patent applications cited herein are incorporated herein by reference in their entirety.

1. A recombinant yeast having an enhanced acetic acid assimilation ability resulting from the introduction of the ac-etaldehyde dehydrogenase gene (EC 1.2.1.10);
having a lowered expression level of a trehalase gene;
having a high expression level of an alcohol dehydrogenase gene having activity of converting acetaldehyde into ethanol; and
having a lowered expression level of an alcohol dehydrogenase gene having activity of converting ethanol into acetaldehyde.

2. The recombinant yeast according to claim 1, wherein the acetaldehyde dehydrogenase gene encodes the acetaldehyde dehydrogenase derived from E. coli.

3. The recombinant yeast according to claim 2, wherein the acetaldehyde dehydrogenase derived from E. coli is the protein (a) or (b) below:
(a) a protein consisting of the amino acid sequence as shown in SEQ ID NO: 2, 4, or 6; or
(b) a protein consisting of an amino acid sequence having 90% or more identity with the amino acid sequence as shown in SEQ ID NO: 2, 4, or 6 and having acetaldehyde dehydrogenase activity.

4. (canceled)

5. The recombinant yeast according to claim 1, which further comprises the xylose isomerase gene introduced thereinto.

6. The recombinant yeast according to claim 5, wherein the xylose isomerase is the protein (a) or (b) below:
(a) a protein consisting of the amino acid sequence as shown in SEQ ID NO: 12; or
(b) a protein consisting of an amino acid sequence having 90% or more identity with the amino acid sequence as shown in SEQ ID NO: 12 and having enzyme activity of converting xylose into xylulose.

7. The recombinant yeast according to claim 1, which further comprises the xylulokinase gene introduced thereinto.

8. The recombinant yeast according to claim 1, which further comprises a gene encoding an enzyme selected from the group of enzymes constituting a nonoxidative process pathway in the pentose phosphate pathway introduced thereinto.

9. The recombinant yeast according to claim 8, wherein the group of enzymes constituting a non-oxidative process pathway in the pentose phosphate pathway includes ribose-5-phosphate isomerase, ribulose¬5-phosphate-3-epimerase, transketolase, and transaldolase.

10.-11. (canceled)

12. A method for producing ethanol comprising a step of ethanol fermentation via culture of the recombinant yeast according to claim 1 in a medium containing glucose and/or xylose.

13. The method according to claim 12, wherein the medium contains cellulose and the ethanol fermentation proceeds simultaneously with saccharification of at least the cellulose.
